SEQUENCE LISTING (177 sequences)

SEQ ID NO: 1 - protein, 15 aa, Felis catus
KEDLLKKLKKWCVIP

SEQ ID NO: 2 - protein, 15 aa, Canis lupus
KDIFLRQIKKWCVVP

SEQ ID NO: 3 - protein, 15 aa, Felis catus
QDTLEKVKLWCSVTK

SEQ ID NO: 4 - protein, 15 aa, Canis lupus
DDALRASTLWCSVST

SEQ ID NO: 5 - protein, 15 aa, Felis catus
SESLRDLEKARRWCV

SEQ ID NO: 6 - protein, 15 aa, Canis lupus
SNKAEDLEKARRWCV

SEQ ID NO: 7 - protein, 15 aa, Felis catus
SFVAYTKDWCTKPKK

SEQ ID NO: 8 - protein, 15 aa, Canis lupus
SFLSYTRDWCTKPKN

SEQ ID NO: 9 - protein, 208 aa, synthetic construct
MSIPLKFLAGAAGVGTVSTGAYFAISHDKGTNIKDRLEASGYSILNLDKGDSEKWEKIKEAYGKEDDEALRFNGVNKNDASTIVGIKTACSSLLASKSESLRDLEKARRWCVVPVTVSSRIGDNLALLSSQDSQDTSLWNAKLEEHKKEASKFTKIPNVWDDKANSDELKLTAFKKKCEEMSKLNTFDKDFESFVAYTKDWCTKPKKG

SEQ ID NO: 10 - protein, 185 aa, synthetic construct
AISHDKGTNIKDRLEASGYSILNLDKGDSEKWEKIKEAYGKEDDEALRFNGVNKNDASTIVGIKTACSSLLASKSESLRDLEKARRWCVVPVTVSSRIGDNLALLSSQDSQDTSLWNAKLEEHKKEASKFTKIPNVWDDKANSDELKLTAFKKKCEEMSKLNTFDKDFESFVAYTKDWCTKPKKG

SEQ ID NO: 11 - protein, 211 aa, synthetic construct
MGSSHHHHHHSSGLVPRGSHMKSPIFNKSKPTLRSEVEGDKWVFLTSRNTEEITKVLNAYKVNSPTVTLKFDGITGNESDASKKLLEKCKELYEKPSDTPSKEDLLKKLKKWCVIPKTVNQRLGDLNYTALSTNGPTQGTTESNDWKQKSQSHTSSSNNKFTGISVTGTGNDDAGAKALREHCKTKLEVNSFSEDFQDTLEKVKLWCSVTK

SEQ ID NO: 12 - protein, 191 aa, synthetic construct
MKSPIFNKSKPTLRSEVEGDKWVFLTSRNTEEITKVLNAYKVNSPTVTLKFDGITGNESDASKKLLEKCKELYEKPSDTPSKEDLLKKLKKWCVIPKTVNQRLGDLNYTALSTNGPTQGTTESNDWKQKSQSHTSSSNNKFTGISVTGTGNDDAGAKALREHCKTKLEVNSFSEDFQDTLEKVKLWCSVTK

SEQ ID NO: 13 - protein, 20 aa, synthetic construct
MGSSHHHHHHSSGLVPRGSH

SEQ ID NO: 14 - protein, 8 aa, synthetic construct
DYKDDDDK

SEQ ID NO: 15 - protein, 10 aa, synthetic construct
YPYDVPDYAC

SEQ ID NO: 16 - protein, 11 aa, synthetic construct
EQKLISEEDLC

SEQ ID NO: 17 - protein, 14 aa, synthetic construct
GKPIPNPLLGLDST

SEQ ID NO: 18 - protein, 13 aa, synthetic construct
GAPVPYPDPLEPR

SEQ ID NO: 19 - protein, 11 aa, synthetic construct
YTDIEMNRLGK

SEQ ID NO: 20 - protein, 6 aa, synthetic construct
HHHHHH

SEQ ID NO: 21 - protein, 12 aa, synthetic construct
QPELAPEDPEDC

SEQ ID NO: 22 - protein, 15 aa, synthetic construct
  SITE 1: note = Residue attached to Biotin via a linker
  SITE 15: note = Amidated residue
MAISHDKGTNIKDRL

SEQ ID NO: 23 - protein, 15 aa, synthetic construct
  SITE 1: note = Residue attached to Biotin via a linker
  SITE 15: note = Amidated residue
DKGTNIKDRLEASGY

SEQ ID NO: 24 - protein, 15 aa, synthetic construct
  SITE 1: note = Residue attached to Biotin via a linker
  SITE 15: note = Amidated residue
IKDRLEASGYSILNL

SEQ ID NO: 25 - protein, 15 aa, synthetic construct
  SITE 1: note = Residue attached to Biotin via a linker
  SITE 15: note = Amidated residue
EASGYSILNLDKGDS

SEQ ID NO: 26 - protein, 15 aa, synthetic construct
  SITE 1: note = Residue attached to Biotin via a linker
  SITE 15: note = Amidated residue
SILNLDKGDSEKWEK

SEQ ID NO: 27 - protein, 15 aa, synthetic construct
  SITE 1: note = Residue attached to Biotin via a linker
  SITE 15: note = Amidated residue
DKGDSEKWEKIKEAY

SEQ ID NO: 28 - protein, 15 aa, synthetic construct
  SITE 1: note = Residue attached to Biotin via a linker
  SITE 15: note = Amidated residue
EKWEKIKEAYGKEDD

SEQ ID NO: 29 - protein, 15 aa, synthetic construct
  SITE 1: note = Residue attached to Biotin via a linker
  SITE 15: note = Amidated residue
IKEAYGKEDDEALRF

SEQ ID NO: 30 - protein, 15 aa, synthetic construct
  SITE 1: note = Residue attached to Biotin via a linker
  SITE 15: note = Amidated residue
GKEDDEALRFNGVNK

SEQ ID NO: 31 - protein, 15 aa, synthetic construct
  SITE 1: note = Residue attached to Biotin via a linker
  SITE 15: note = Amidated residue
EALRFNGVNKNDAST

SEQ ID NO: 32 - protein, 15 aa, synthetic construct
  SITE 1: note = Residue attached to Biotin via a linker
  SITE 15: note = Amidated residue
NGVNKNDASTIVGIK

SEQ ID NO: 33 - protein, 15 aa, synthetic construct
  SITE 1: note = Residue attached to Biotin via a linker
  SITE 15: note = Amidated residue
NDASTIVGIKTACSS

SEQ ID NO: 34 - protein, 15 aa, synthetic construct
  SITE 1: note = Residue attached to Biotin via a linker
  SITE 15: note = Amidated residue
IVGIKTACSSLLASK

SEQ ID NO: 35 - protein, 15 aa, synthetic construct
  SITE 1: note = Residue attached to Biotin via a linker
  SITE 15: note = Amidated residue
TACSSLLASKSESLR

SEQ ID NO: 36 - protein, 15 aa, synthetic construct
  SITE 1: note = Residue attached to Biotin via a linker
  SITE 15: note = Amidated residue
LLASKSESLRDLEKA

SEQ ID NO: 37 - protein, 15 aa, synthetic construct
  SITE 1: note = Residue attached to Biotin via a linker
  SITE 15: note = Amidated residue
DLEKARRWCVVPVTV

SEQ ID NO: 38 - protein, 15 aa, synthetic construct
  SITE 1: note = Residue attached to Biotin via a linker
  SITE 15: note = Amidated residue
RRWCVVPVTVSSRIG

SEQ ID NO: 39 - protein, 15 aa, synthetic construct
  SITE 1: note = Residue attached to Biotin via a linker
  SITE 15: note = Amidated residue
VPVTVSSRIGDNLAL

SEQ ID NO: 40 - protein, 15 aa, synthetic construct
  SITE 1: note = Residue attached to Biotin via a linker
  SITE 15: note = Amidated residue
SSRIGDNLALLSSQD

SEQ ID NO: 41 - protein, 15 aa, synthetic construct
  SITE 1: note = Residue attached to Biotin via a linker
  SITE 15: note = Amidated residue
DNLALLSSQDSQDTS

SEQ ID NO: 42 - protein, 15 aa, synthetic construct
  SITE 1: note = Residue attached to Biotin via a linker
  SITE 15: note = Amidated residue
LSSQDSQDTSLWNAK

SEQ ID NO: 43 - protein, 15 aa, synthetic construct
  SITE 1: note = Residue attached to Biotin via a linker
  SITE 15: note = Amidated residue
SQDTSLWNAKLEEHK

SEQ ID NO: 44 - protein, 15 aa, synthetic construct
  SITE 1: note = Residue attached to Biotin via a linker
  SITE 15: note = Amidated residue
LWNAKLEEHKKEASK

SEQ ID NO: 45 - protein, 15 aa, synthetic construct
  SITE 1: note = Residue attached to Biotin via a linker
  SITE 15: note = Amidated residue
LEEHKKEASKFTKIP

SEQ ID NO: 46 - protein, 15 aa, synthetic construct
  SITE 1: note = Residue attached to Biotin via a linker
  SITE 15: note = Amidated residue
KEASKFTKIPNVWDD

SEQ ID NO: 47 - protein, 15 aa, synthetic construct
  SITE 1: note = Residue attached to Biotin via a linker
  SITE 15: note = Amidated residue
FTKIPNVWDDKANSD

SEQ ID NO: 48 - protein, 15 aa, synthetic construct
  SITE 1: note = Residue attached to Biotin via a linker
  SITE 15: note = Amidated residue
NVWDDKANSDELKLT

SEQ ID NO: 49 - protein, 15 aa, synthetic construct
  SITE 1: note = Residue attached to Biotin via a linker
  SITE 15: note = Amidated residue
KANSDELKLTAFKKK

SEQ ID NO: 50 - protein, 15 aa, synthetic construct
  SITE 1: note = Residue attached to Biotin via a linker
  SITE 15: note = Amidated residue
ELKLTAFKKKCEEMS

SEQ ID NO: 51 - protein, 15 aa, synthetic construct
  SITE 1: note = Residue attached to Biotin via a linker
  SITE 15: note = Amidated residue
AFKKKCEEMSKLNTF

SEQ ID NO: 52 - protein, 15 aa, synthetic construct
  SITE 1: note = Residue attached to Biotin via a linker
  SITE 15: note = Amidated residue
CEEMSKLNTFDKDFE

SEQ ID NO: 53 - protein, 15 aa, synthetic construct
  SITE 1: note = Residue attached to Biotin via a linker
  SITE 15: note = Amidated residue
KLNTFDKDFESFVAY

SEQ ID NO: 54 - protein, 15 aa, synthetic construct
  SITE 1: note = Residue attached to Biotin via a linker
  SITE 15: note = Amidated residue
DKDFESFVAYTKDWC

SEQ ID NO: 55 - protein, 11 aa, synthetic construct
  SITE 1: note = Residue attached to Biotin via a linker
  SITE 11: note = Amidated residue
TKDWCTKPKKG

SEQ ID NO: 56 - protein, 15 aa, synthetic construct
AESLRDLEKARRWCV

SEQ ID NO: 57 - protein, 15 aa, synthetic construct
SASLRDLEKARRWCV

SEQ ID NO: 58 - protein, 15 aa, synthetic construct
SEALRDLEKARRWCV

SEQ ID NO: 59 - protein, 15 aa, synthetic construct
SESARDLEKARRWCV

SEQ ID NO: 60 - protein, 15 aa, synthetic construct
SESLADLEKARRWCV

SEQ ID NO: 61 - protein, 15 aa, synthetic construct
SESLRALEKARRWCV

SEQ ID NO: 62 - protein, 15 aa, synthetic construct
SESLRDAEKARRWCV

SEQ ID NO: 63 - protein, 15 aa, synthetic construct
SESLRDLAKARRWCV

SEQ ID NO: 64 - protein, 15 aa, synthetic construct
SESLRDLEAARRWCV

SEQ ID NO: 65 - protein, 15 aa, synthetic construct
WVFLTSRNTEEITKV

SEQ ID NO: 66 - protein, 15 aa, synthetic construct
SESLRDLEKAARWCV

SEQ ID NO: 67 - protein, 15 aa, synthetic construct
SESLRDLEKARAWCV

SEQ ID NO: 68 - protein, 15 aa, synthetic construct
SESLRDLEKARRACV

SEQ ID NO: 69 - protein, 15 aa, synthetic construct
SESLRDLEKARRWAV

SEQ ID NO: 70 - protein, 15 aa, synthetic construct
SESLRDLEKARRWCA

SEQ ID NO: 71 - protein, 15 aa, synthetic construct
AFVAYTKDWCTKPKK

SEQ ID NO: 72 - protein, 15 aa, synthetic construct
SAVAYTKDWCTKPKK

SEQ ID NO: 73 - protein, 15 aa, synthetic construct
SFAAYTKDWCTKPKK

SEQ ID NO: 74 - protein, 10 aa, synthetic construct
DLEKARRWCV

SEQ ID NO: 75 - protein, 15 aa, synthetic construct
SFVAATKDWCTKPKK

SEQ ID NO: 76 - protein, 15 aa, synthetic construct
SFVAYAKDWCTKPKK

SEQ ID NO: 77 - protein, 15 aa, synthetic construct
SFVAYTADWCTKPKK

SEQ ID NO: 78 - protein, 15 aa, synthetic construct
SFVAYTKAWCTKPKK

SEQ ID NO: 79 - protein, 15 aa, synthetic construct
SFVAYTKDACTKPKK

SEQ ID NO: 80 - protein, 15 aa, synthetic construct
SFVAYTKDWATKPKK

SEQ ID NO: 81 - protein, 15 aa, synthetic construct
SFVAYTKDWCAKPKK

SEQ ID NO: 82 - protein, 15 aa, synthetic construct
SFVAYTKDWCTAPKK

SEQ ID NO: 83 - protein, 15 aa, synthetic construct
SFVAYTKDWCTKAKK

SEQ ID NO: 84 - protein, 15 aa, synthetic construct
SFVAYTKDWCTKPAK

SEQ ID NO: 85 - protein, 15 aa, synthetic construct
SFVAYTKDWCTKPKA

SEQ ID NO: 86 - protein, 15 aa, synthetic construct
AEDLLKKLKKWCVIP

SEQ ID NO: 87 - protein, 15 aa, synthetic construct
KADLLKKLKKWCVIP

SEQ ID NO: 88 - protein, 15 aa, synthetic construct
KEALLKKLKKWCVIP

SEQ ID NO: 89 - protein, 15 aa, synthetic construct
KEDALKKLKKWCVIP

SEQ ID NO: 90 - protein, 15 aa, synthetic construct
KEDLAKKLKKWCVIP

SEQ ID NO: 91 - protein, 15 aa, synthetic construct
KEDLLAKLKKWCVIP

SEQ ID NO: 92 - protein, 15 aa, synthetic construct
KEDLLKALKKWCVIP

SEQ ID NO: 93 - protein, 15 aa, synthetic construct
KEDLLKKAKKWCVIP

SEQ ID NO: 94 - protein, 15 aa, synthetic construct
KEDLLKKLAKWCVIP

SEQ ID NO: 95 - protein, 15 aa, synthetic construct
KEDLLKKLKAWCVIP

SEQ ID NO: 96 - protein, 15 aa, synthetic construct
KEDLLKKLKKACVIP

SEQ ID NO: 97 - protein, 15 aa, synthetic construct
KEDLLKKLKKWAVIP

SEQ ID NO: 98 - protein, 15 aa, synthetic construct
KEDLLKKLKKWCAIP

SEQ ID NO: 99 - protein, 15 aa, synthetic construct
KEDLLKKLKKWCVAP

SEQ ID NO: 100 - protein, 15 aa, synthetic construct
KEDLLKKLKKWCVIA

SEQ ID NO: 101 - protein, 15 aa, synthetic construct
ADTLEKVKLWCSVTK

SEQ ID NO: 102 - protein, 15 aa, synthetic construct
QATLEKVKLWCSVTK

SEQ ID NO: 103 - protein, 15 aa, synthetic construct
QDALEKVKLWCSVTK

SEQ ID NO: 104 - protein, 15 aa, synthetic construct
QDTAEKVKLWCSVTK

SEQ ID NO: 105 - protein, 15 aa, synthetic construct
QDTLAKVKLWCSVTK

SEQ ID NO: 106 - protein, 15 aa, synthetic construct
QDTLEAVKLWCSVTK

SEQ ID NO: 107 - protein, 15 aa, synthetic construct
QDTLEKAKLWCSVTK

SEQ ID NO: 108 - protein, 15 aa, synthetic construct
QDTLEKVALWCSVTK

SEQ ID NO: 109 - protein, 15 aa, synthetic construct
QDTLEKVKAWCSVTK

SEQ ID NO: 110 - protein, 15 aa, synthetic construct
QDTLEKVKLACSVTK

SEQ ID NO: 111 - protein, 15 aa, synthetic construct
QDTLEKVKLWASVTK

SEQ ID NO: 112 - protein, 15 aa, synthetic construct
QDTLEKVKLWCAVTK

SEQ ID NO: 113 - protein, 15 aa, synthetic construct
QDTLEKVKLWCSATK

SEQ ID NO: 114 - protein, 15 aa, synthetic construct
QDTLEKVKLWCSVAK

SEQ ID NO: 115 - protein, 15 aa, synthetic construct
QDTLEKVKLWCSVTA

SEQ ID NO: 116 - protein, 19 aa, synthetic construct
MDWTWRILFLVAAATGTHA

SEQ ID NO: 117 - protein, 22 aa, Erwinia sp.
MKYLLPTAAAGLLLLAAQPAMA

SEQ ID NO: 118 - protein, 25 aa, Homo sapiens
MATGSRTSLLLAFGLLCLWLQEGSA

SEQ ID NO: 119 - protein, 16 aa, synthetic construct
MPLLLLLPLLWAGALA

SEQ ID NO: 120 - protein, 35 aa, Homo sapiens
MDAMKRGLCCVLLLCGAVFVSLSQEIHAELRRFRR

SEQ ID NO: 121 - protein, 21 aa, synthetic construct
MKQSTIALALLPLLFTPVKTA

SEQ ID NO: 122 - protein, 21 aa, synthetic construct
MKKTAIAIAVALAGFATVAQA

SEQ ID NO: 123 - protein, 19 aa, synthetic construct
MKKIWLALAGLVLAFSASA

SEQ ID NO: 124 - protein, 17 aa, synthetic construct
MRVLLFLLSLFMLPAFS

SEQ ID NO: 125 - protein, 27 aa, synthetic construct
MSLSRRQFIQASGIALCAGAVPLKASA

SEQ ID NO: 126 - protein, 39 aa, synthetic construct
MNNNDLFQASRRRFLAQLGGLTVAGMLGPSLLTPRRATA

SEQ ID NO: 127 - protein, 30 aa, synthetic construct
MAGPATQSPMKLMALQLLLWHSALWTVQEA

SEQ ID NO: 128 - protein, 25 aa, Homo sapiens
MMPSSVSWGILLLAGLCCLVPVSLA

SEQ ID NO: 129 - protein, 46 aa, Homo sapiens
MQRVNMIMAESPSLITICLLGYLLSAECTVFLDHENANKILNRPKR

SEQ ID NO: 130 - protein, 21 aa, Homo sapiens
MKGSLLLLLVSNLLLCQSVAP

SEQ ID NO: 131 - protein, 24 aa, Homo sapiens
MKWVTFISLLFLFSSAYSRGVFRR

SEQ ID NO: 132 - protein, 99 aa, synthetic construct
MVSLKIKKLLLVSLLNAIEAYSNDTIYSTSYNNGIESTPSYSTSAISSTGSSNKENAITSSSETTTMAGDYGESGSTTIMDEQETGTSSQYISVTTTTQ

SEQ ID NO: 133 - protein, 43 aa, synthetic construct
NGGNMAIKKASLIALLPLFTAAAAAATDAETSNESGSSSHLKS

SEQ ID NO: 134 - protein, 84 aa, synthetic construct
MKFTSVLAFFLATLTASATFLYKRQNVTSGGGTVPIITGGPAVSGSQSNVTTTTLFNSTSTLNITQLYQIATDVNDTLQSESSS

SEQ ID NO: 135 - protein, 100 aa, synthetic construct
MQFPFACLLSTLVISGSLARASPFDFIFGNGTQQAQSQSESQGQVSFTNEASQDSSTTSLVTAYSQGVHSHQSATIVSATISSLPSTWYDASSTSQTSVS

SEQ ID NO: 136 - protein, 96 aa, synthetic construct
MQFKNALTATAILSASALAANSTTSIPSSCSIGTSATATAQADLDKISQCSTIVGNLTITGDLGSAALASIQEIDGSLTIFNSSSLSSFSADIKKI

SEQ ID NO: 137 - protein, 77 aa, synthetic construct
MKFSTLSTVAAIAAFASADSTSDGVTYVDVTTTPQSTTSMVSTVKTTSTPYTTSTIATLSTKSISSQANTTTHEIST

SEQ ID NO: 138 - protein, 100 aa, synthetic construct
MFVHRLWTLAFPFLVEISKASQLENIKSLLDIEDNVLPNLNISQNNSNAVQILGGVDALSFYEYTGQQNFTKEIGPETSSHGLVYYSNNTYIQLEDASDD

SEQ ID NO: 139 - protein, 66 aa, synthetic construct
MKLQSLLVSAAVLTSLTENVNAMSPNNSYVPANVTCDDDINLVREASGLSDNEYEMLKKRDAYTKE

SEQ ID NO: 140 - protein, 10 aa, Felis catus
SFVAYTKDWC

SEQ ID NO: 141 - protein, 10 aa, Felis catus
KKLKKWCVIP

SEQ ID NO: 142 - protein, 215 aa, synthetic construct
MTTLTKAAAGMGALGTAAGGGVLLSKSPIFNKSKPTLRSEVEGDKWVFLTSRNTEEITKVLNAYKVNSPTVTLKFDGITGNESDASKKLLEKCKELYEKPSDTPSKEDLLKKLKKWCVIPKTVNQRLGDLNYTALSTNGPTQGTTESNDWKQKSQSHTSSSNNKFTGISVTGTGNDDAGAKALREHCKTKLEVNSFSEDFQDTLEKVKLWCSVTK

SEQ ID NO: 143 - protein, 15 aa, synthetic construct
DDAGAKALREHCKTK

SEQ ID NO: 144 - protein, 15 aa, synthetic construct
DGITGNESDASKKLL

SEQ ID NO: 145 - protein, 15 aa, synthetic construct
EITKVLNAYKVNSPT

SEQ ID NO: 146 - protein, 15 aa, synthetic construct
EKCKELYEKPSDTPS

SEQ ID NO: 147 - protein, 15 aa, synthetic construct
ESNDWKQKSQSHTSS

SEQ ID NO: 148 - protein, 15 aa, synthetic construct
FSEDFQDTLEKVKLW

SEQ ID NO: 149 - protein, 15 aa, synthetic construct
HCKTKLEVNSFSEDF

SEQ ID NO: 150 - protein, 15 aa, synthetic construct
KALREHCKTKLEVNS

SEQ ID NO: 151 - protein, 15 aa, synthetic construct
KKLKKWCVIPKTVNQ

SEQ ID NO: 152 - protein, 15 aa, synthetic construct
KQKSQSHTSSSNNKE

SEQ ID NO: 153 - protein, 15 aa, synthetic construct
KSPIFNKSKPTLRSE

SEQ ID NO: 154 - protein, 15 aa, synthetic construct
KTVNQRLGDLNYTAL

SEQ ID NO: 155 - protein, 15 aa, synthetic construct
LEVNSFSEDFQDTLE

SEQ ID NO: 156 - protein, 15 aa, synthetic construct
LNAYKVNSPTVTLKF

SEQ ID NO: 157 - protein, 15 aa, synthetic construct
LYEKPSDTPSKEDLL

SEQ ID NO: 158 - protein, 15 aa, synthetic construct
NESDASKKLLEKCKE

SEQ ID NO: 159 - protein, 15 aa, synthetic construct
NKSKPTLRSEVEGDK

SEQ ID NO: 160 - protein, 15 aa, synthetic construct
NYTALSTNGPTQGTT

SEQ ID NO: 161 - protein, 15 aa, synthetic construct
RLGDLNYTALSTNGP

SEQ ID NO: 162 - protein, 15 aa, synthetic construct
SDTPSKEDLLKKLKK

SEQ ID NO: 163 - protein, 15 aa, synthetic construct
SHTSSSNNKFTGISV

SEQ ID NO: 164 - protein, 15 aa, synthetic construct
SKKLLEKCKELYEKP

SEQ ID NO: 165 - protein, 15 aa, synthetic construct
SNNKFTGISVTGTGN

SEQ ID NO: 166 - protein, 15 aa, synthetic construct
SRNTEEITKVLNAYK

SEQ ID NO: 167 - protein, 15 aa, synthetic construct
STNGPTQGTTESNDW

SEQ ID NO: 168 - protein, 15 aa, synthetic construct
TGISVTGTGNDDAGA

SEQ ID NO: 169 - protein, 15 aa, synthetic construct
TGTGNDDAGAKALRE

SEQ ID NO: 170 - protein, 16 aa, synthetic construct
TLRSEVEGGDKWVFLT

SEQ ID NO: 171 - protein, 15 aa, synthetic construct
TQGTTESNDWKQKSQ

SEQ ID NO: 172 - protein, 15 aa, synthetic construct
VEGDKWVFLTSRNTE

SEQ ID NO: 173 - protein, 15 aa, synthetic construct
VNSPTVTLKEDGITG

SEQ ID NO: 174 - protein, 15 aa, synthetic construct
VTLKFDGITGNESDA

SEQ ID NO: 175 - protein, 15 aa, synthetic construct
WCVIPKTVNQRLGDL

SEQ ID NO: 176 - protein, 15 aa, synthetic construct
  SITE 1: note = Residue attached to Biotin via a linker
  SITE 15: note = Amidated residue
SESLRDLEKARRWCV

SEQ ID NO: 177 - protein, 15 aa, synthetic construct
  SITE 1: note = Residue attached to Biotin via a linker
  SITE 15: note = Amidated residue
SFVAYTKDWCTKPKK

(19) United States
(12) Patent Application Publication
(10) Pub. No.: US 2024/0280575 A1
(43) Pub. Date: Aug. 22, 2024

(54) Compositions and Methods for the Detection of Mycoplasma

(71) Applicant: IDEXX Laboratories, Inc., Westbrook, ME (US)

(72) Inventors: Raghavan PALANIAPPAN, West Des Moines, IA (US); Ethan Everett Pope, Westbrook, ME (US); Maha Yerramilli, Scarborough, ME (US)

(73) Assignee: IDEXX Laboratories, Inc., Westbrook, ME (US)

(21) Appl. No.: 18/401,086

(22) Filed: Dec. 29, 2023

Related U.S. Application Data
(60) Provisional application No. 63/477,850, filed on Dec. 30, 2022.

(51) Int. Cl. G01N 33/569; C07K 14/30

(52) U.S. Cl. CPC G01N 33/56933; C07K 14/30; G01N 33/56983; G01N 2333/15; G01N 2333/155; G01N 2333/30; G01N 2469/20

(57) ABSTRACT

Provided herein are compositions and methods for detection of detection and treatment of Mycoplasma haemofelis and Mycoplasma haemocanis.

### PRIORITY

This application claims the benefit of U.S. Ser. No. 63/477,850, which was filed on Dec. 30, 2023, and is incorporated by reference herein in its entirety.

### SEQUENCE LISTING

The instant application contains a Sequence Listing which has been submitted electronically in XML file format and is hereby incorporated by reference in its entirety. Said XML copy, created on Jan. 11, 2024, is named 748743_IDX-012_SL.xml and is 184,349 bytes in size.

### BACKGROUND

Hemotrophic mycoplasmas (hemoplasmas) are uncultivable cell-wall less bacteria (previously known as Haemobartonella and Eperythrozoon species), that adhere to the surface of erythrocytes in vertebrate hosts. Hemotrophic mycoplasmas are a clade within the Mycoplasma genus and are phylogenetically related to the pneumoniae group of the mycoplasmas.

Mycoplasma haemocanis is a hemotrophic mycoplasma blood pathogen that can cause acute disease in immunosuppressed or splenectomized dogs. Cats can be infected with Mycoplasma haemofelis causing subclinical infection and hemolytic anemia. Mycoplasma haemofelis and Mycoplasma haemocanis infections have traditionally been detected by cytologic evaluation of blood smears to detect the pleomorphic bacteria on the erythrocytes of the host. Cytological detection of Mycoplasma haemofelis often provides false-positives due to artifacts and false-negatives due to varying parasitemia levels in sick cats. PCR assays be difficult to implement in veterinary practices due to specialized equipment needs and quality control issues. Better and more accessible detection methods are needed in the art.

Anemia is one of the common hematological abnormalities found in infected cats. Mycoplasma haemofelis (Mhf), feline leukemia virus (FeLV) and feline-immunodeficiency virus (FIV) are the causative agents for feline infectious anemia and fever. It is important for clinicians to differentiate viral agents such as FIV and FeLV from the bacterial agent such as Mhf for definite treatment.

Current diagnosis relies on PCR with whole blood samples. Since the infection is mostly of cyclic pattern, the presence of Mycoplasma after infection may present in lower or undetectable amount in the blood. Furthermore, RBCs are replenished in every cycle and the infection appears to be cleared but mycoplasma can reappear.

The maximum copy number following infection with M. haemofelis occurs around 14-15 days post-infection (DPI) (10^{8.6}-10^{9.6} copies/ml of blood) but with large variation in M. haemofelis copy number over time. These fluctuations in copy number can be as large and as rapid as a 4 log difference over 2 or 3 days, or a 7 log difference over 12 days.

### BRIEF DESCRIPTION OF THE FIGURES

FIG. 1 shows an indirect ELISA assay for the detection anti-M. haemofelis antibodies in sera samples using rTDX1362.

FIG. 2 shows a diagram of TDX1362 (SEQ ID NO:9).

FIG. 3 shows the testing of B4 peptides, C11 peptides, and mature TDX1362 polypeptides with M. haemofelis negative sera samples.

FIG. 4A-4C shows the testing of B4 peptides, C11 peptides, and mature TDX1362 polypeptides with M. haemofelis positive sera samples.

FIG. 5A-5B shows the results from an indirect ELISA antibody assay for point mutation alanine scanning. SESLRDLEKARRWCV is SEQ ID NO:5; SFVAYTKDACTKPKK is SEQ ID NO:79; SESLRDLEKARAWCV is SEQ ID NO:67; SFVAYTKDWCTKPKA is SEQ ID NO:85.

FIG. 6 shows and indirect ELISA assay with recombinant TDX1452 used to test for antibody response in M. haemofelis experimentally infected cats over time.

FIG. 7 shows the detection of M. haemofelis in experimentally infected cats using labeled polyclonal antibodies specific for TDX1452.

FIG. 8 shows epitope mapping by alanine scanning of the B5 and C12 peptides.

FIG. 9 shows a diagram of TDX1452 (SEQ ID NO: 142).

FIG. 10 shows the use of a series of 15-mer 10 overlapping peptides of TDX1452 to elucidate the immunodominant regions of the protein. DDAGAKALREHCKTK is SEQ ID NO:143; DGITGNESDASKKLL is SEQ ID NO:144; EITKVLNAYKVNSPT is SEQ ID NO:145, EKCKELYEKPSDTPS is SEQ ID NO. 146; ESNDWKQKSQSHTSS is SEQ ID NO:147; FSEDFQDTLEKVKLW is SEQ ID NO: 148; HCKTKLEVNSFSEDF is SEQ ID NO:149; KALREHCKTKLEVNS is SEQ ID NO: 150; KEDLLKKLKKWCVIP is SEQ ID NO:1; KKLKKWCVIPKTVNQ is SEQ ID NO:151; KQKSQSHTSSSNNKEF is SEQ ID NO:152; KSPIFNKSKPTLRSE is SEQ ID NO 153; KTVNQRLGDLNYTAL is SEQ ID NO:154; LEVNSFSEDFQDTLE is SEQ ID NO:155; LNAYKVNSPTVTLKF is SEQ ID NO:156; LYEKPSDTPSKEDLL is SEQ ID NO:157; NESDASKKLLEKCKE is SEQ ID NO:158; NKSKPTLRSEVEGDK is SEQ ID NO:159; NYTALSTNGPTQGTT is SEQ ID NO:160; QDTLEKVKLWCSVTK is SEQ ID NO:3; RLGDLNYTALSTNGP is SEQ ID NO:161; SDTPSKEDLLKKLKK is SEQ ID NO:162; SHTSSSNNKFTGISV is SEQ ID NO:163; SKKLLEKCKELYEKP is SEQ ID NO:164; SNNKFTGISVTGTGN is SEQ ID NO:165; SRNTEEITKVLNAYK is SEQ ID NO:166; STNGPTQGTTESNDW is SEQ ID NO:167; TGISVTGTGNDDAGA is SEQ ID NO:168; TGTGNDDAGAKALRE is SEQ ID NO: 169; TLRSEVEGGDKWVFLT is SEQ ID NO: 170; TQGTTESNDWKQKSQ is SEQ ID NO:171; VEGDKWVFLTSRNTE is SEQ ID NO:172; VNSPTVTLKEDGITG is SEQ ID NO:173; VTLKFDGITGNESDA is SEQ ID NO:174; WCVIPKTVNQRLGDL is SEQ ID NO:175; WVFLTSRNTEEITKV is SEQ ID NO:65.

### SUMMARY

Provided herein are one or more polypeptides, e.g., purified polypeptides, comprising one or more of:
(i) a polypeptide as set forth in SEQ ID NO:1, 2, 3, 4, 5, 6, 7, 8, 37, 54, 74, 97, 111, 140, 141, or 151;
(ii) a polypeptide comprising SEQ ID NO:1, wherein:
   (a) Wat position 11 is retained and 1, 2, or 3 other amino acids are substituted with a conservative amino acid substitution;
   (b) W at position 11 is retained and conservative amino acid substitutions are at 1, 2, or 3 of the following positions: 1, 2, 3, 4, 5, 6, 7, 8, 9, 10, 12, 13, 14, or 15; or
   (c) W at position 11 is retained and conservative amino acid substitutions are at 1, 2, or 3 of the following positions: 2, 3, 4, 6, 7, 8, or 14;
iii) a polypeptide comprising SEQ ID NO:2, wherein:
   (a) W at position 11 is retained and 1, 2, 3 other amino acids are substituted with a conservative amino acid substitution;
   (b) W at position 11 is retained and conservative amino acid substitutions are at 1, 2, or 3 of the following positions: 1, 2, 3, 4, 5, 6, 7, 8, 9, 10, 12, 13, 14, or 15; or
   (c) W at position 11 is retained and conservative amino acid substitutions are at 1, 2, or 3 of the following positions: 2, 3, 4, 6, 7, 8, or 14;
(iv) a polypeptide comprising SEQ ID NO:3 wherein:
   (a) W at position 10 is retained and 1, 2, 3 other amino acids are substituted with a conservative amino acid substitution;
   (b) W at position 10 is retained and conservative amino acid substitutions are at 1, 2, or 3 of the following positions: 1, 2, 3, 4, 5, 6, 7, 8, 9, 11, 12, 13, 14, or 15; or
   (c) W at position 11 is retained and conservative amino acid substitutions are at 1, 2, or 3 of the following positions: 1, 3, 5, 6, 7, 8, 14 or 15
(v) a polypeptide comprising SEQ ID NO:4, wherein:
   (a) W at position 10 is retained and 1, 2, 3 other amino acids are substituted with a conservative amino acid substitution;
   (b) W at position 10 is retained and conservative amino acid substitutions are at 1, 2, or 3 of the following positions: 1, 2, 3, 4, 5, 6, 7, 8, 9, 11, 12, 13, 14, or 15; or
   (c) W at position 11 is retained and conservative amino acid substitutions are at 1, 2, or 3 of the following positions: 1, 3, 5, 6, 7, 8, 14 or 15;
(vi) a polypeptide comprising SEQ ID NO:5, wherein:
   (a) K at position 9 and Wat position 13 are retained and 1, 2, 3 other amino acids are substituted with a conservative amino acid substitution;
   (b) K at position 9 and W at position 13 are retained and conservative amino acid substitutions are at 1, 2, or 3 of the following positions: 1, 2, 3, 4, 5, 6, 7, 8, 10, 11, 12, 14, or 15; or
   (c) K at position 9 and W at position 13 are retained and conservative amino acid substitutions are at 1, 2, or 3 of the following positions: 2, 3, 4, or 5;
(vii) a polypeptide comprising SEQ ID NO:6, wherein:
   (a) K at position 9 and the W at position 13 are retained and 1, 2, 3 other amino acids are substituted with a conservative amino acid substitution;
   (b) K at position 9 and W at position 13 are retained and conservative amino acid substitutions are at 1, 2, or 3 of the following positions: 1, 2, 3, 4, 5, 6, 7, 8, 10, 11, 12, 14, or 15; or
   (c) K at position 9 and W at position 13 are retained and conservative amino acid substitutions are at 1, 2, or 3 of the following positions: 2, 3, 4, or 5.
(viii) a polypeptide comprising SEQ ID NO:7, wherein:
   (a) K at position 7, D at position 8, and W at position 9 are retained and 1, 2, 3 other amino acids are substituted with a conservative amino acid substitution;
   (b) K at position 7, D at position 8, and W at position 9 are retained and conservative amino acid substitutions are at 1, 2, or 3 of the following positions: 1, 2, 3, 4, 5, 6, 10, 11, 12, 13, 14, or 15; or
   (c) Kat position 7, D at position 8, and Wat position 9 are retained and conservative amino acid substitutions are at 1, 2, or 3 of the following positions: 3, 4, or 15.
(ix) a polypeptide comprising SEQ ID NO:8, wherein:
   (a) D at position 8 and W at position 9 are retained and 1, 2, 3 other amino acids are substituted with a conservative amino acid substitution;
   (b) D at position 8 and W at position 9 are retained and conservative amino acid substitutions are at 1, 2, or 3 of the following positions: 1, 2, 3, 4, 5, 6, 7, 10, 11, 12, 13, 14, or 15; or
   (c) D at position 8 and the W at position 9 are retained and conservative amino acid substitutions are at 1, 2, or 3 of the following positions: 3, 4, 7, or 15;
(x) a polypeptide comprising SEQ ID NO:10;
(xi) a polypeptide having 95% or more sequence identity to SEQ ID NO: 10, wherein amino acids 75-89, 170-184, or 75-89, and 170-184 are retained;
(xii) a polypeptide comprising SEQ ID NO:12;
(xiii) a polypeptide having 95% or more sequence identity to SEQ ID NO:12, wherein amino acids 82-96, 177-191, or 82-96 and 177-191 are retained;
(xiv) a polypeptide comprising SEQ ID NO:9 or SEQ ID NO:11;
(xv) a polypeptide comprising a polypeptide comprising SEQ ID NO: 141, wherein:
   (a) W at position 6 is retained and 1, 2, or 3 other amino acids are substituted with a conservative amino acid substitution;
   (b) W at position 6 is retained and conservative amino acid substitutions are at 1, 2, or 3 of the following positions: 1, 2, 3, 4, 5, 7, 8, 9, or 10; or
   (c) W at position 6 is retained and position 7 is substituted with A, and optionally conservative amino acid substitutions are at 1, 2, or 3 of the following positions: 1, 2, 3, 4, 5, 8, 9, or 10;
(xvi) a polypeptide comprising a polypeptide comprising SEQ ID NO:151, wherein:
   (a) W at position 6 is retained and 1, 2, or 3 other amino acids are substituted with a conservative amino acid substitution;
   (b) W at position 6 is retained and conservative amino acid substitutions are at 1, 2, or 3 of the following positions: 1, 2, 3, 4, 5, 7, 8, 9, 10, 11, 12, 13, or 14; or
   (c) W at position 6 is retained and position 7 is substituted with A, and optionally conservative amino acid substitutions are at 1, 2, or 3 of the following positions: 1, 2, 3, 4, 5, 8, 9, 10, 11, 12, 13, or 14;
(xvii) a polypeptide comprising a polypeptide comprising SEQ ID NO:97, wherein:
   (a) W at position 11 is retained and 1, 2, or 3 other amino acids are substituted with a conservative amino acid substitution;
   (b) W at position 11 is retained and conservative amino acid substitutions are at 1, 2, or 3 of the following positions: 1, 2, 3, 4, 5, 6, 7, 8, 9, 10, 12, 13, 14, or 15; or
   (c) W at position 11 is retained and position 12 is substituted with A, and optionally conservative amino acid substitutions are at 1, 2, or 3 of the following positions: 1, 2, 3, 4, 5, 8, 9, 10, 13, 14, or 15;
(xviii) a polypeptide comprising SEQ ID NO:74, wherein:
   (a) K at position 4 and W at position 8 are retained and 1, 2, 3 other amino acids are substituted with a conservative amino acid substitution; or
   (b) K at position 4 and W at position 8 are retained and conservative amino acid substitutions are at 1, 2, or 3 of the following positions: 1, 2, 3, 5, 6, 7, or 10;
(xix) a polypeptide comprising SEQ ID NO:37, wherein:
   (a) K at position 4 and W at position 8 are retained and 1, 2, 3 other amino acids are substituted with a conservative amino acid substitution; or
   (b) K at position 4 and W at position 8 are retained and conservative amino acid substitutions are at 1, 2, or 3 of the following positions: 1, 2, 3, 5, 6, 7, 11, 12, 13, 14, or 15;
(xx) a polypeptide comprising SEQ ID NO:54, wherein:
   (a) K at position 12, D at position 13, and W at position 14 are retained and 1, 2, 3 other amino acids are substituted with a conservative amino acid substitution;
   (b) K at position 12, D at position 13, and W at position 14 are retained and conservative amino acid substitutions are at 1, 2, or 3 of the following positions: 1, 2, 3, 4, 5, 6, 7, 8, 9, 10, 11, or 15; or
   (c) K at position 12, D at position 13, and W at position 14 are retained and conservative amino acid substitutions are at 1, 2, or 3 of the following positions: 7 or 8.
(xxi) a polypeptide comprising SEQ ID NO:140, wherein:
   (a) K at position 7, D at position 8, and W at position 9 are retained and 1, 2, 3 other amino acids are substituted with a conservative amino acid substitution;
   (b) K at position 7, D at position 8, and W at position 9 are retained and conservative amino acid substitutions are at 1, 2, or 3 of the following positions: 1, 2, 3, 4, 5, 6, or 10; or
   (c) K at position 7, D at position 8, and W at position 9 are retained and conservative amino acid substitutions are at 1, 2, or 3 of the following positions: 3 or 4;
(xxii) a polypeptide comprising SEQ ID NO:111 wherein:
   (a) W at position 10 is retained and 1, 2, 3 other amino acids are substituted with a conservative amino acid substitution;
   (b) W at position 10 is retained and conservative amino acid substitutions are at 1, 2, or 3 of the following positions: 1, 2, 3, 4, 5, 6, 7, 8, 9, 11, 12, 13, 14, or 15; or
(c) W at position 11 is retained and conservative amino acid substitutions are at 1, 2, 3 of the following positions: 1, 3, 5, 6, 7, 8, 14 and 15.

These polypeptides can be present in a fusion protein comprising two, three, four, five, six, seven or more polypeptides having 90% or more sequence identity to the one or more polypeptides described herein.

Another aspect provides a polypeptide having less than 190 total amino acids and comprising 90% or more sequence identity to a polypeptide as set forth in SEQ ID NO:10 or 12. In some aspects one or more polypeptides described herein are not naturally occurring. The one or more polypeptides can be lyophilized, desiccated, or dried. The one or more polypeptides can comprise one or more labels or tags. The one or more polypeptides can be immobilized to a support. The one or more polypeptides can be present in an immunocomplex with one or more antibodies that specifically bind to a polypeptide as set forth in SEQ ID NO:1, 2, 3, 4, 5, 6, 7, 8, 9, 10, 11, 12, 37, 54, 74, 97, 111, 140, 141, or 151. The one or more polypeptides can comprise one or more secretory signal sequences, one or more epitope tags, or one or more secretory signal sequences and one or more epitope tags.

Another aspect provides a method of detecting anti-Mycoplasma haemofelis or anti-Mycoplasma haemocanis antibodies or specific binding fragments thereof. The method comprises (a) contacting a test sample with the one or more polypeptides described herein; and (b) detecting complexes of anti-Mycoplasma haemofelis or anti-Mycoplasma haemocanis antibodies or specific binding fragments thereof and the one or more polypeptides. The one or more polypeptides can be immobilized to a support. The complexes can be detected using one or more secondary antibodies or specific binding fragments thereof that specifically bind anti-Mycoplasma haemofelis or anti-Mycoplasma haemocanis antibodies or specific binding fragments thereof. The secondary antibodies or specific binding fragments thereof can comprise one or more tags or labels. The complexes can be detected using one or more detector polypeptides wherein the one or more detector polypeptides are the one or more polypeptides described herein (e.g., the polypeptides of claim 1 or 3). The one or more detector polypeptides can comprise a label or tag.

Another aspect provides a method for diagnosing a disease caused by Mycoplasma haemofelis or Mycoplasma haemocanis antibodies in a subject by (a) contacting a test sample with the one or more polypeptides described herein (e.g., the polypeptides of claim 1 or claim 3); and (b) detecting complexes of anti-Mycoplasma haemofelis or anti-Mycoplasma haemocanis antibodies or specific binding fragments thereof and the one or more polypeptides. The subject can have been infected with Mycoplasma haemofelis or Mycoplasma haemocanis for less than 15 days. The method can further comprise comparing an amount of the complexes in the sample to a control sample or control standard, wherein elevated levels of the complexes as compared to the control sample or control standard is an indication of a disease caused by Mycoplasma haemofelis or Mycoplasma haemocanis. The method can further comprise administering a treatment for a disease caused by Mycoplasma haemofelis or Mycoplasma haemocanis where the complexes are detected. The method can further comprise determining an amount of the anti-Mycoplasma haemofelis or anti-Mycoplasma haemocanis antibodies or specific binding fragments thereof in the sample. The subject can be a non-human animal. The test sample can be blood, plasma, serum, or lymph fluid.

In some aspects the methods provide for the detection of anti-Mycoplasma haemofelis or anti-Mycoplasma haemocanis antibodies or specific binding fragments thereof by a competitive immunoassay, a sandwich immunoassay, an enzyme-linked immunosorbent assay (ELISA), an immunohistochemical assay, a turbidimetric immunoassay, a particle-enhanced turbidimetric immunoassay, a radioimmunoassay (RIA), a fluorescent immunosorbent assay (FIA), a multiplex immunoassay, a protein/peptide array immunoassay, a solid phase radioimmunoassay (SPRIA), an indirect immunofluorescence assay (IIF), a chemiluminescent immunoassay (CIA), a particle based multianalyte test (PMAT), a dot blot assay, a western blot assay, surface plasmon resonance (SPR), isothermal titration calorimetry (ITC), microscale thermophoresis (MST), biolayer interferometry, or grating-coupled interferometry. A multiplex assay can additionally test for feline leukemia virus (FeLV), feline-immunodeficiency virus (FIV), or both FeLV and FIV.

Yet another aspect provides a kit for diagnosing a disease caused by Mycoplasma haemofelis or Mycoplasma haemocanis, the kit. The kit can comprise
(a) one or more polypeptides described herein (e.g., polypeptides of claim 1 or claim 3), wherein the polypeptide is not naturally occurring; and
(b) one or more reagents that facilitate binding of the one or more polypeptides to anti Mycoplasma haemofelis or anti-Mycoplasma haemocanis antibodies or specific binding fragments thereof present in a test sample.

An aspect provides a method for treating a disease caused by Mycoplasma haemofelis or Mycoplasma haemocanis antibodies in a subject by (a) contacting a test sample with the one or more polypeptides described herein; (b) detecting complexes of anti-Mycoplasma haemofelis or anti-Mycoplasma haemocanis or specific binding fragments thereof and the one or more polypeptides; (c) treating the subject with one or more antibiotics where complexes of anti-Mycoplasma haemofelis or anti-Mycoplasma haemocanis antibodies or specific binding fragments thereof and the one or more polypeptides are detected. The subject can have been infected with Mycoplasma haemofelis or Mycoplasma haemocanis for less than 15 days (e.g., 15, 14, 13, 12, 11, 10, 9, 8, 7 or less days). An amount of the complexes in the sample to a control sample or control standard can be determined, wherein elevated levels of the complexes as compared to the control sample or control standard is an indication of a disease caused by Mycoplasma haemofelis or Mycoplasma haemocanis. An amount of the anti-Mycoplasma haemofelis or anti-Mycoplasma haemocanis antibodies or specific binding fragments thereof can be determined in the sample. The subject can be a non-human animal. The test sample can be blood, plasma, serum, or lymph fluid. The anti-Mycoplasma haemofelis or anti-Mycoplasma haemocanis antibodies or specific binding fragments thereof can be detected by a competitive immunoassay, a sandwich immunoassay, an enzyme-linked immunosorbent assay (ELISA), an immunohistochemical assay, a turbidimetric immunoassay, a particle-enhanced turbidimetric immunoassay, a radioimmunoassay (RIA), a fluorescent immunosorbent assay (FIA), a multiplex immunoassay, a protein/peptide array immunoassay, a solid phase radioimmunoassay (SPRIA), an indirect immunofluorescence assay (IIF), a chemiluminescent immunoassay (CIA), a particle based multianalyte test (PMAT), a dot blot assay, a western blot assay, surface plasmon resonance (SPR), isothermal titration calorimetry (ITC), microscale thermophoresis (MST), biolayer interferometry, or grating-coupled interferometry.

Another aspect provides a polynucleotide encoding the one or more polypeptides or fusion proteins disclosed herein.

### DETAILED DESCRIPTION

Provided herein are compositions and methods for detection of detection and treatment of Mycoplasma haemofelis and Mycoplasma haemocanis.

### Polypeptides

A polypeptide is a polymer where amide bonds covalently link three or more amino acids. A polypeptide can be post-translationally modified. A purified polypeptide is a polypeptide preparation that is substantially free of cellular material, other types of poly peptides, chemical precursors, chemicals used in synthesis of the polypeptide, or combinations thereof. A polypeptide preparation that is substantially free of cellular material, culture medium, chemical precursors, chemicals used in synthesis of the polypeptide has less than about 30%, 20%, 10%, 5%, 1% or less of other polypeptides, culture medium, chemical precursors, and/or other chemicals used in synthesis. Therefore, a purified polypeptide is about 70%, 80%, 90%, 95%, 99% or more pure.

The term “polypeptides” can refer to one or more types of polypeptides or a set of polypeptides. “Polypeptides” can also refer to mixtures of two or more different types of polypeptides including, but not limited to, full-length proteins, truncated polypeptides, or polypeptide fragments. The term “polypeptides” or “polypeptide” can each mean “one or more polypeptides.”

In some aspects a polypeptide comprises a feline TDX1452 B5 epitope: KEDLLKKLKKWCVIP (SEQ ID NO:1). In some aspects a TDX1452 polypeptide comprises a canine TDX1452 B5 epitope: KDIFLRQIKKWCVVP (SEQ ID NO:2). In some aspects a TDX1452 polypeptide comprises a feline TDX1452 C12 epitope: QDTLEKVKLWCSVTK (SEQ ID NO:3). In some aspects a TDX1452 polypeptide comprises a canine TDX1452 C12 epitope: DDALRASTLWCSVST (SEQ ID NO:4). The feline and canine epitopes are aligned below.

TDX1452 epitopes aligned:

| | B5 Feline |
|---|---|
| (SEQ ID NO: 1) | |
| | KEDLLKKLKKWCVIP |
| | B5 Canine |
| (SEQ ID NO: 2) | |
| | KDIFLRQIKKWCVVP |
| | C12 Feline |
| (SEQ ID NO: 3) | |
| | QDTLEKVKLWCSVTK |
| | C12 Canine |
| (SEQ ID NO: 4) | |
| | DDALRASTLWCSVST |

It is expected that antibodies specific for Mycoplasma haemofelis and Mycoplasma haemocanis will retain specific binding to the polypeptides having conservative amino acid substitutions because, inter alia, the experiments in Examples 3-5 demonstrate that specific binding can be retained when amino acid substitutions are made.

In some aspects a polypeptide comprises a TDX1452 feline B5 epitope comprising SEQ ID NO:1 wherein the W at position 11 is retained and 1, 2, 3, 4, 5, or more other amino acids are substituted with a conservative amino acid substitution. In an aspect, the W at position 11 is retained and the conservative amino acid substitutions are at 1, 2, 3, 4, 5, or more of the following positions: 1, 2, 3, 4, 5, 6, 7, 8, 9, 10, 12, 13, 14, or 15. In an aspect, the Wat position 11 is retained and the conservative amino acid substitutions are at 1, 2, 3, 4, 5, or more of the following positions: 2, 3, 4, 6, 7, 8, or 14.

In some aspects a polypeptide comprises a TDX1452 canine B5 epitope comprising SEQ ID NO:2 wherein the W at position 11 is retained and 1, 2, 3, 4, 5, or more other amino acids are substituted with a conservative amino acid substitution. In an aspect, the Wat position 11 is retained and the conservative amino acid substitutions are at 1, 2, 3, 4, 5, or more of the following positions: 1, 2, 3, 4, 5, 6, 7, 8, 9, 10, 12, 13, 14, or 15. In an aspect, the W at position 11 is retained and the conservative amino acid substitutions are at 1, 2, 3, 4, 5, or more of the following positions: 2, 3, 4, 6, 7, 8, or 14.

In some aspects a polypeptide comprises a TDX1452 feline C12 epitope comprising SEQ ID NO:3 wherein the W at position 10 is retained and 1, 2, 3, 4, 5, or more other amino acids are substituted with a conservative amino acid substitution. In an aspect, the Wat position 10 is retained and the conservative amino acid substitutions are at 1, 2, 3, 4, 5, or more of the following positions: 1, 2, 3, 4, 5, 6, 7, 8, 9, 11, 12, 13, 14, or 15. In an aspect, the W at position 11 is retained and the conservative amino acid substitutions are at 1, 2, 3, 4, 5, or more of the following positions: 1, 3, 5, 6, 7, 8, 14 or 15.

In some aspects a polypeptide comprises a TDX1452 canine C12 epitope comprising SEQ ID NO:4 wherein the W at position 10 is retained and 1, 2, 3, 4, 5, or more other amino acids are substituted with a conservative amino acid substitution. In an aspect, the Wat position 10 is retained and the conservative amino acid substitutions are at 1, 2, 3, 4, 5, or more of the following positions: 1, 2, 3, 4, 5, 6, 7, 8, 9, 11, 12, 13, 14, or 15. In an aspect, the W at position 11 is retained and the conservative amino acid substitutions are at 1, 2, 3, 4, 5, or more of the following positions: 1, 3, 5, 6, 7, 8, 14 or 15.

In some aspects a polypeptide comprises a feline TDX1362 B4 epitope: SESLRDLEKARRWCV (SEQ ID NO:5). In some aspects a TDX1362 polypeptide comprises a canine TDX1362 B4 epitope: SNKAEDLEKARRWCV (SEQ ID NO:6). In some aspects a polypeptide comprises a feline TDX1362 C11 epitope: SFVAYTKDWCTKPKK (SEQ ID NO:7). In some aspects a polypeptide comprises a canine TDX1362 C11 epitope: SFLSYTRDWCTKPKN (SEQ ID NO:8). The feline and canine epitopes are aligned below:

| | B4 Feline |
|---|---|
| (SEQ ID NO: 5) | |
| | SESLRDLEKARRWCV |
| | B4 Canine |
| (SEQ ID NO: 6) | |
| | SNKAEDLEKARRWCV |
| | C11 Feline |
| (SEQ ID NO: 7) | |
| | SFVAYTKDWCTKPKK |
| | C11 Canine |
| (SEQ ID NO: 8) | |
| | SFLSYTRDWCTKPKN |

In some aspects a polypeptide comprises a TDX1362 feline B4 epitope comprising SEQ ID NO:5 wherein the K at position 9 and the W at position 13 are retained and 1, 2, 3, 4, 5, or more other amino acids are substituted with a conservative amino acid substitution. In an aspect, the K at position 9 and the W at position 13 are retained and the conservative amino acid substitutions are at 1, 2, 3, 4, 5, or more of the following positions: 1, 2, 3, 4, 5, 6, 7, 8, 10, 11, 12, 14, or 15. In an aspect, the K at position 9 and the W at position 13 are retained and the conservative amino acid substitutions are at 1, 2, 3, 4, 5, or more of the following positions: 2, 3, 4, or 5.

In some aspects a polypeptide comprises a TDX1362 canine B4 epitope comprising SEQ ID NO:6 wherein the K at position 9 and the W at position 13 are retained and 1, 2, 3, 4, 5, or more other amino acids are substituted with a conservative amino acid substitution. In an aspect, the K at position 9 and the W at position 13 are retained and the conservative amino acid substitutions are at 1, 2, 3, 4, 5, or more of the following positions: 1, 2, 3, 4, 5, 6, 7, 8, 10, 11, 12, 14, or 15. In an aspect, the K at position 9 and the W at position 13 are retained and the conservative amino acid substitutions are at 1, 2, 3, 4, 5, or more of the following positions: 2, 3, 4, or 5.

In some aspects a polypeptide comprises a TDX1362 feline C11 epitope comprising SEQ ID NO:7 wherein the K at position 7, the D at position 8, and the W at position 9 are retained and 1, 2, 3, 4, 5, or more other amino acids are substituted with a conservative amino acid substitution. In an aspect, the K at position 7, the D at position 8, and the W at position 9 are retained and the conservative amino acid substitutions are at 1, 2, 3, 4, 5, or more of the following positions: 1, 2, 3, 4, 5, 6, 10, 11, 12, 13, 14, or 15. In an aspect, the K at position 7, the D at position 8, and the W at position 9 are retained and the conservative amino acid substitutions are at 1, 2, 3, 4, 5, or more of the following positions: 3, 4, or 15.

In some aspects a polypeptide comprises a TDX1362 canine C11 epitope comprising SEQ ID NO:8 wherein the D at position 8 and the Wat position 9 are retained and 1, 2, 3, 4, 5, or more other amino acids are substituted with a conservative amino acid substitution. In an aspect, the D at position 8 and the W at position 9 are retained and the conservative amino acid substitutions are at 1, 2, 3, 4, 5, or more of the following positions: 1, 2, 3, 4, 5, 6, 7, 10, 11, 12, 13, 14, or 15. In an aspect, the D at position 8 and the W at position 9 are retained and the conservative amino acid substitutions are at 1, 2, 3, 4, 5, or more of the following positions: 3, 4, 7, or 15.

In some aspects a polypeptide comprises a TDX1362 polypeptide:

(SEQ ID NO: 9)
MSIPLKFLAGAAGVGTVSTGAYFAISHDKGTNIKDRLEASGYSILNIDKG
DSEKWEKIKEAYGKEDDEALRENGVNKNDASTIVGIKTACSSLLASKSES
LRDLEKARRWCVVPVTVSSRIGDNLALLSSODSQDTSLWNAKLEEHKKEA
SKFTKIPNVWDDKANSDELKLTAFKKKCEEMSKLNTEDKDFESFVAYTKD
WCTKPKKG

In some aspects a polypeptide comprises a TDX1362 polypeptide without a signal sequence:

(SEQ ID NO: 10)
AISHDKGTNIKDRLEASGYSILNIDKGDSEKWEKIKEAYGKEDDEALREN
GVNKNDASTIVGIKTACSSLLASKSESLRDLEKARRWCVVPVTVSSRIGD
NLALLSSQDSQDTSLWNAKLEEHKKEASKFTKIPNVWDDKANSDELKLTA
FKKKCEEMSKLNTEDKDFESFVAYTKDWCTKPKKG.

In an aspect, a variant TDX1362 polypeptide is provided. A variant can comprise polypeptide having 80, 85, 90, 93, 95, 96, 97, 98, 99% or more sequence identity to SEQ ID NO:10, wherein amino acids 75-89, 170-184, or 75-89, and 170-184 (bolded above) are retained, i.e., not substituted or deleted.

In certain aspects, a polypeptide comprises a TDX1452 polypeptide linked to an MGS Hiss-tag (SEQ ID NO: 20)/thrombin cleavage site:

(SEQ ID NO: 11)
MGSSHHHHHHSSGLVPRGSHMKSPIFNKSKPTLRSEVEGDKWVFLTSRN
TEEITKVLNAYKVNSPTVTLKFDGITGNESDASKKLLEKCKELYEKPSD
TPSKEDLLKKLKKWCVIPKTVNQRLGDLNYTALSTNGPTQGTTESNDWK
QKSQSHTSSSNNKFTGISVTGTGNDDAGAKALREHCKTKLEVNSFSEDFQ
DTLEKVKLWCSVTK

The MGS Hise-tag (SEQ ID NO: 20)/thrombin cleavage site is

(SEQ ID NO: 13)
MGSSHHHHHHSSGLVPRGSH.

A TDX1452 polypeptide without a MGS Hiss-tag (SEQ ID NO: 20)/thrombin cleavage site is shown below.

(SEQ ID NO: 12)
MKSPIFNKSKPTLRSEVEGDKWVFLTSRNTEEITKVLNAY
KVNSPTVTLKFDGITGNESDASKKLLEKCKELYEKPSDTPSKEDLLKKL
KKWCVIPKTVNQRLGDLNYTALSTNGPTQGTTESNDWKQKSQSHTSSSN
NKFTGISVTGTGNDDAGAKALREHCKTKLEVNSFSEDFQDTLEKVKLWC
SVTK.

In an aspect, a variant TDX1452 polypeptide is provided. A variant can comprise polypeptide having 80, 85, 90, 93, 95, 96, 97, 98, 99% or more sequence identity to SEQ ID NO:12, wherein amino acids 82-96, 177-191, or 82-96 and 177-191 (bolded above) are retained, i.e., not substituted or deleted.

In an aspect a polypeptide comprises two or more (e.g., 2, 3, 4, 5, 6, 7 or more) of SEQ ID NOs: 1-12) fused or linked together. In an aspect a polypeptide can be a multimer, that is, a polypeptide can comprise 2, 3, 4, 5, 6, 7, 8, 9, 10, or more copies of one of SEQ ID NO: 1 to SEQ ID NO: 12 fused or linked together. In an aspect a polypeptide comprises one or more of a label, a tag, a signal sequence, a cleavage site (e.g., a thrombin cleavage site or a enterokinase cleavage site), solid support (e.g., a bead or membrane)

Any of these polypeptides can be immobilized to a support such as, for example, barcoded magnetic beads (BMBs) and used in a detection assay.

Any one of the polypeptides or variant polypeptides described above can be non-naturally occurring. In some aspects one or more amino acid substitutions, deletions, or additions can change any of SEQ ID NOs: 1-12 to a non-naturally occurring polypeptide.

A secretory signal sequence is a peptide sequence (or polynucleotide encoding the peptide sequence) present at the N-terminus (or in some cases, the C-terminus) of a peptide sequence. A secretory signal sequence can be referred to by various names such as, but not limited to, a signal sequence, a targeting signal, a localization signal, a localization sequence, a transit peptide, a leader sequence, a leader peptide, a prepro sequence, a pre sequence, or a secretory signal peptide). A secretory signal sequence can be about 10 to 110 amino acids long (e.g., about 10, 15, 20, 25, 30, 35, 40, 45, 50, 55, 60, 65, 70, 75, 80, 85, 90, 95, 100, 105 or 110 amino acids long). A secretory signal sequence, as a component of a larger polypeptide, can be useful for targeting and can direct the larger polypeptide through a secretory pathway of a cell in which it is synthesized. In some embodiments, the larger polypeptide is cleaved to remove the secretory signal sequence during transit through the secretory pathway. A secretory signal sequence can be endogenous or engineered.

A secretory signal sequence can be synthesized according to the rules established, for example, by von Heinje (Eur. J. Biochem. 133: 17-21, 1983; J. Mol. Biol. 184: 99-105, 1985; Nuc. Acids. Res. 14: 4683-3690, 1986). Examples of secretory signal sequences are shown in Table 1. Any secretory signal sequence known in the art or to those of ordinary skill in the art can be used in the polypeptides and methods described herein.

TABLE 1

| Description | | |
|---|---|---|
| (if applicable) | A secretory signal sequence | SEQ ID NO: |
| SignalP 3.0 | MDWTWRILFLVAAATGTHA | SEQ ID NO: 116 |
| Erwinia carotovora | MKYLLPTAAAGLLLLAAQPAMA | SEQ ID NO: 117 |
| pectate lyase 2 | | |
| (pelB) leader | | |
| peptide | | |
| Human growth | MATGSRTSLLLAFGLLCLWLQEGSA | SEQ ID NO: 118 |
| hormone signal | | |
| sequence | | |
| otPA pre-pro | MPLLLLLPLLWAGALA | SEQ ID NO: 119 |
| signal sequence | | |
| Human CD33 | MDAMKRGLCCVLLLCGAVFVSLSQEIHAELR | SEQ ID NO: 120 |
| signal sequence | RFRR | |
| PhoA | MKQSTIALALLPLLFTPVKTA | SEQ ID NO: 121 |
| OmpA | MKKTAIAIAVALAGFATVAQA | SEQ ID NO: 122 |
| DsbA | MKKIWLALAGLVLAFSASA | SEQ ID NO: 123 |
| TorT | MRVLLFLLSLFMLPAFS | SEQ ID NO: 124 |
| SufI | MSLSRRQFIQASGIALCAGAVPLKASA | SEQ ID NO: 125 |
| TorA | MNNNDLFQASRRRFLAQLGGLTVAGMLGPS | SEQ ID NO: 126 |
| | LLTPRRATA | |
| N terminal | MAGPATQSPMKLMALQLLLWHSALWTVQEA | SEQ ID NO: 127 |
| secretory signal | | |
| peptide sequence | | |
| human α-1-anti | MMPSSVSWGILLLAGLCCLVPVSLA | SEQ ID NO: 128 |
| trypsin (AAT) | | |
| human Factor IX | MQRVNMIMAESPSLITICLLGYLLSAECTVFLD | SEQ ID NO: 129 |
| (FIX) | HENANKILNRPKR | |
| human Prolactin | MKGSLLLLLVSNLLLCQSVAP | SEQ ID NO: 130 |
| (Prolac) | | |
| human Albumin | MKWVTFISLLFLFSSAYSRGVFRR | SEQ ID NO: 131 |
| (Alb) | | |
| YDR420w | MVSLKIKKLLLVSLLNAIEAYSNDTI | SEQ ID NO: 132 |
| secretory signal | YSTSYNNGIESTPSYSTSAISSTGSSNKENAIT | |
| peptide | SSSETTTMAGDYGESGSTTIMDEQETGTSSQ | |
| | YISVTTTTQ | |
| YBR187w | NGGNMAIKKASLIALLPLFTAAAAAATDAETSN | SEQ ID NO: 133 |
| secretory signal | ESGSSSHLKS | |
| peptide | | |
| YHR139c | MKFTSVLAFFLATLTASATFLYKRQNVTSGGG | SEQ ID NO: 134 |
| secretory signal | TVPIITGGPAVSGSQSNVTTTTLFNSTSTLNIT | |
| peptide | QLYQIATDVNDTLQSESSS | |
| YGR014w | MQFPFACLLSTLVISGSLARASPFDFIFGNGT | SEQ ID NO: 135 |
| secretory signal | QQAQSQSESQGQVSFTNEASQDSSTTSLVT | |
| peptide | AYSQGVHSHQSATIVSATISSLPSTWYDASST | |
| | SQTSVS | |
| YBR078w | MQFKNALTATAILSASALAANSTTSIPSSCSIG | SEQ ID NO: 136 |
| secretory signal | TSATATAQADLDKISQCSTIVGNLTITGDLGSA | |
| peptide | ALASIQEIDGSLTIFNSSSLSSFSADIKKI | |
| YNL300w | MKFSTLSTVAAIAAFASADSTSDGVTYVDVTT | SEQ ID NO: 137 |
| secretory signal | TPQSTTSMVSTVKTTSTPYTTSTIATLSTKSIS | |
| peptide | SQANTTTHEIST | |
| YLR084c | MFVHRLWTLAFPFLVEISKASQLENIKSLLDIE | SEQ ID NO: 138 |
| secretory signal | DNVLPNLNISQNNSNAVQILGGVDALSFYEYT | |
| peptide | GQQNFTKEIGPETSSHGLVYYSNNTYIQLEDA | |
| | SDD | |
| YMR008c | MKLQSLLVSAAVLTSLTENVNAMSPNNSYVP | SEQ ID NO: 139 |
| secretory signal | ANVTCDDDINLVREASGLSDNEYEMLKKRDA | |
| peptide | YTKE | |

A polypeptide can comprise about 70%, 75%, 80%, 85%, 90%, 95%, 96%, 97%, 98%, 99% or more sequence identity to one or more polypeptides (e.g., 1, 2, 3, 4, 5, 6, 7, or more) as set forth in SEQ ID NOs:1-12 and can further comprise one or more (e.g., 1, 2, 3, 4, 5, or more) secretory signal sequences, one or more (e.g., 1, 2, 3, 4, 5, or more) epitope tags, or one or more secretory signal sequences and one or more epitope tags. These elements can be present as a fusion protein with one or more linkers between the individual proteins or sequences making up the fusion protein. Alternatively, no linkers can be present between the individual proteins or sequences making up the fusion protein. The elements of the fusion protein (i.e., a polypeptide as set forth in SEQ ID NOs: 1-12, secretory signal sequences, and epitope tags) can occur in any order in the fusion protein. These polypeptides are therefore non-naturally occurring and have different properties than naturally occurring polypeptides including, for example, a reduced tendency to form secondary structures and the ability to conjugate to solid phases for testing.

Certain aspects provide a purified polypeptide comprising SEQ ID NO:1-12 or a fragment thereof. A polypeptide fragment can be less than about 50, 40, 30, 20, 15, 14, 13 or 12 amino acids total contiguous amino acids.

In certain aspects a polypeptide fragment is more than about 5, 8, 9, 10, 11, 12, 13, 14, 15, 20, 25, 30, 35, 40, 50, 60, 70, 80, 90, 95, 100, 150, 200, or 210 total contiguous amino acids of SEQ ID NO: 1-12. In an embodiment, a polypeptide has less than about 95, 75, 50, 25, or 10 total amino acids and comprises 70%, 80%, 90%, 95% or more sequence identity to a polypeptide as set forth in SEQ ID NO:1-12. In an embodiment a polypeptide has less than about 200, 190, 180,170, 160, 150, 100, 95, 90, 80, 70, 60, 50, 40, 35, 25, 20, 15, 14, 13, 12, 11, 10, 9, 8, or 5 total amino acids and comprises about 70%, 80%, 90%, 95% or more sequence identity to a polypeptide as set forth in SEQ ID NO:1-12.

For example, SEQ ID NO:9 is 208 amino acids in length. In an embodiment, the polypeptide can comprise additional amino acids to extend the polypeptide to a length of, for example, 250 amino acids. The extra or additional amino acids can be, for example, labels, tags, additional Mycoplasma haemofelis amino acids, amino acids unrelated to Mycoplasma haemofelis, amino acids that can be used for purification, amino acids that can be used to increase solubility of the polypeptide, amino acids to improve other characteristics of the polypeptide, or other amino acids. In an embodiment, the additional amino acids are not Mycoplasma haemofelis amino acids. In this example, the 250 amino acid long polypeptide has about 70%, 80%, 90%, 95% or more sequence identity to a polypeptide as set forth in SEQ ID NO:9 over the 208 consecutive amino acids of SEQ ID NO:9, while the remaining 42 amino acids of the 208 amino acid polypeptide can have, e.g., no sequence identity to SEQ ID NO:9.

The fact that a polypeptide (e.g., SEQ ID NOs: 1-8) is smaller than a full length a Mycoplasma haemofelis or Mycoplasma haemocanis polypeptide can be important because smaller polypeptides can have greater specificity and/or sensitivity than full length polypeptides in detection or diagnostic assays. Additionally, these smaller polypeptides can be less expensive to manufacture and can be obtained at greater purity than full length polypeptides.

In one embodiment, a polypeptide or fragment thereof is non-naturally occurring. That is, a polypeptide or fragment comprises 1, 2, 3, 4, 5, 10, 20, 30, 40, 50, 75 or more non-naturally occurring amino acids. In an embodiment, the non-naturally occurring amino acids can provide a beneficial property such as increased solubility of the polypeptide or increased sensitivity or increased specificity of the polypeptide in assays.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g., SEQ ID NOs:1-12) aligned for comparison purposes is at least 50, 60, 70, or 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 83%, at least 85%, at least 90%, at least 95%, at least 98%, at least 99%, at least 99.5%, or at least 99.9%. In general, an exact match indicates 100% identity over the length of the reference sequence (e.g., SEQ ID NOs:1-12).

Polypeptides that are sufficiently similar to polypeptides described herein can be used herein. Polypeptides that are about 90, 91, 92, 93, 94 95, 96, 97, 98, 99, 99.5% or more identical to polypeptides described herein can also be used herein.

A polypeptide variant differs by about, for example, 1, 2, 3, 4, 5, 6, 7, 8, 9, 10, 20, 30, 40, 50, 60, 70, 80, 90, or more amino acid residues (e.g., amino acid additions, substitutions, or deletions) from a peptide shown SEQ ID NOs: 1-12 or a fragment thereof. Where this comparison requires alignment, the sequences are aligned for maximum sequence identity. The site of variation can occur anywhere in the polypeptide. In one embodiment, a variant has about 70%, 75%, 80%, 85%, 90%, 95%, 96%, 97%, 98%, 99% sequence identity to the original polypeptide.

Variant polypeptides can generally be identified by modifying one of the polypeptide sequences described herein and evaluating the properties of the modified polypeptide to determine if it is a biological equivalent. A variant is a biological equivalent if it reacts substantially the same as a polypeptide described herein in an assay such as an immunohistochemical assay, an enzyme-linked immunosorbent assay (ELISA), a turbidimetric immunoassay, a particle-enhanced turbidimetric immunoassay, a particle-enhanced turbidimetric immunoassay, a radioimmuno-assay (RIA), immunoenzyme assay, a western blot assay, or other suitable assay. In other words, a variant is a biological equivalent if it has 90-110% of the activity of the original polypeptide. In one embodiment, the assay is a competition assay wherein the biologically equivalent polypeptide is capable of reducing binding of the polypeptide described herein to a corresponding reactive antigen or antibody by about 80%, 95%, 99%, or 100%. An antibody that specifically binds a corresponding polypeptide also specifically binds the variant polypeptide.

Variant polypeptides can have one or more conservative amino acid variations or other minor modifications and retain biological activity, i.e., are biologically functional equivalents to SEQ ID NOs:1-12 or a fragment thereof. Variant polypeptides can have labels, tags, additional Mycoplasma haemofelis or Mycoplasma haemocanis amino acids, amino acids unrelated to Mycoplasma haemofelis, or Mycoplasma haemocanis amino acids that can be used for purification, amino acids that can be used to increase solubility of the polypeptide, amino acids to improve other characteristics of the polypeptide, or other amino acids. In an embodiment, the additional amino acids are not Mycoplasma haemofelis or Mycoplasma haemocanis amino acids.

Methods of introducing a mutation into an amino acid sequence are well known to those skilled in the art. See, e.g., Ausubel (ed.), Current Protocols in Molecular Biology, John Wiley and Sons, Inc. (1994); Maniatis et al., Molecular Cloning: A Laboratory Manual, Cold Spring Harbor laboratory, Cold Spring Harbor, N.Y. (1989). Mutations can also be introduced using commercially available kits such as “QuikChange™ Site-Directed Mutagenesis Kit” (Stratagene). The generation of a functionally active variant polypeptide by replacing an amino acid that does not influence the function of a polypeptide can be accomplished by one skilled in the art. A variant polypeptide can also be chemically synthesized.

Variant polypeptides can have conservative amino acid substitutions at one or more predicted nonessential amino acid residues. A conservative substitution is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. In one embodiment a polypeptide has about 1, 2, 3, 4, 5, 10, 20 or fewer conservative amino acid substitutions.

A polypeptide can be a fusion protein, which can contain other amino acid sequences, such as amino acid linkers, amino acid spacers, signal sequences, TMR stop transfer sequences, transmembrane domains, as well as ligands useful in protein purification, such as glutathione-S-transferase, histidine tag (e.g., about 6, 7, 8, 9, 10, or more His residues), and staphylococcal protein A, or combinations thereof. In an embodiment, a polypeptide comprises one or more epitope tags, such as FLAG (for example, DYKDDDDK; SEQ ID NO:14), HA (YPYDVPDYAC; SEQ ID NO:15), myc (EQKLISEEDLC; SEQ ID NO:16), V5 (GKPIPNPLLGLDST; SEQ ID NO:17), E-tag (GAPVPYPDPLEPR; SEQ ID NO:18), VSV-g (YTDIEMNRLGK; SEQ ID NO:19), 6xHis (HHHHHHH; SEQ ID NO:20), and HSV (QPELAPEDPEDC; SEQ ID NO:21). An antibody, such as a monoclonal antibody, can specifically bind to an epitope tag and be used to purify a polypeptide comprising the epitope tag.

A fusion protein can comprise two or more different amino acid sequences operably linked to each other. A fusion protein construct can be synthesized chemically using organic compound synthesis techniques by joining individual polypeptide fragments together in fixed sequence. A fusion protein can also be chemically synthesized. A fusion protein construct can also be expressed by a genetically modified host cell (such as E. coli) cultured in vitro, which carries an introduced expression vector bearing specified recombinant DNA sequences encoding the amino acids residues in proper sequence. The heterologous polypeptide can be fused, for example, to the N-terminus or C-terminus of a polypeptide. More than one polypeptide can be present in a fusion protein. Fragments of polypeptides can be present in a fusion protein. A fusion protein can comprise, e.g., one, two, three, four, five, six, seven or more of SEQ ID NOs:1-12, fragments thereof, variants thereof, or combinations thereof. Polypeptides can be in a multimeric form. In other words, a polypeptide can comprise two or more copies (e.g., two, three, four, five, six, seven or more) of SEQ ID NOs:1-12, fragments thereof, variants thereof, or a combination thereof. A polypeptide can include, e.g., a fusion protein of two, three, four, five, six, seven or more polypeptides having about 70%, 75%, 80%, 85%, 90%, 95%, 96%, 97%, 98%, 99% or more sequence identity to SEQ ID NOs: 1-12; or a fusion protein of at least two polypeptides having about 70%, 75%, 80%, 85%, 90%, 95%, 96%, 97%, 98%, 99% sequence identity to SEQ ID NOs: 1-12. A polypeptide can be a fusion protein that can include one or more linkers between the individual proteins making up the fusion protein (i.e., SEQ ID NOs: 1-12). Alternatively, no linkers can be present between the individual proteins making up the fusion protein. A fusion polypeptide can contain other amino acid sequences, such as amino acid linkers, amino acid spacers, signal sequences, TMR stop transfer sequences, transmembrane domains, as well as ligands useful in protein purification, such as glutathione-S-transferase, histidine tag, epitope tags, and staphylococcal protein A, or combinations thereof.

A still further component of a fusion protein can be a secretory (signal) sequence. These sequences can allow for secretion of the fusion protein from the host cell during expression. The secretory (signal) sequence can be that of the heterologous protein being produced, if it has such a sequence, or can be derived from another secreted protein (e.g., t-PA), or synthesized de novo. The polynucleotide sequence encoding the secretory (signal) sequence can be operably linked to fusion protein DNA sequence, i.e., the two sequences are joined in the correct reading frame and positioned to direct the newly synthesized polypeptide into the secretory pathway of the host cell. Polynucleotide sequences encoding secretory (signal) sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

In an embodiment a polypeptide as described herein is present in an immunocomplex with one or more antibodies or specific binding fragments thereof that specifically bind to a polypeptide as set forth in SEQ ID NOs:1-12. The one or more antibodies or specific binding fragments thereof can be anti-Mycoplasma haemofelis antibodies, anti-Mycoplasma haemocanis antibodies, or specific binding fragments thereof.

Polypeptides (e.g., SEQ ID NOs: 1-12) can be lyophilized, desiccated, or dried, for example freeze-dried. A lyophilized polypeptide can be obtained by subjecting a preparation of the polypeptides to low temperatures to remove water from the sample. A desiccated polypeptide composition can be obtained by drying out a preparation of the polypeptides by removal of water. A dried polypeptide preparation can refer to a polypeptide preparation that has been air dried (e.g., lyophilized).

### Polynucleotides

Polynucleotides contain less than an entire microbial genome and can be single- or double-stranded nucleic acids. A polynucleotide can be RNA, mRNA, DNA, cDNA, genomic DNA, chemically synthesized RNA or DNA or combinations thereof. A polynucleotide can comprise, for example, a gene, open reading frame, non-coding region, or regulatory element.

A gene is any polynucleotide molecule that encodes a polypeptide, protein, or fragments thereof, optionally including one or more regulatory elements preceding (5′ non-coding sequences) and following (3′ non-coding sequences) the coding sequence. In one embodiment, a gene does not include regulatory elements preceding and following the coding sequence. A native or wild-type gene refers to a gene as found in nature, optionally with its own regulatory elements preceding and following the coding sequence. A chimeric or recombinant gene refers to any gene that is not a native or wild-type gene, optionally comprising regulatory elements preceding and following the coding sequence, wherein the coding sequences and/or the regulatory elements, in whole or in part, are not found together in nature. Thus, a chimeric gene or recombinant gene comprise regulatory elements and coding sequences that are derived from different sources, or regulatory elements and coding sequences that are derived from the same source but arranged differently than is found in nature. A gene can encompass full-length gene sequences (e.g., as found in nature and/or a gene sequence encoding a full-length polypeptide or protein) and can also encompass partial gene sequences (e.g., a fragment of the gene sequence found in nature and/or a gene sequence encoding a protein or fragment of a polypeptide or protein). A gene can include modified gene sequences (e.g., modified as compared to the sequence found in nature). Thus, a gene is not limited to the natural or full-length gene sequence found in nature.

Polynucleotides can be purified free of other components, such as proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide. Polynucleotides can encode the polypeptides described herein, (e.g., SEQ ID NO:1-12).

Polynucleotides can comprise additional heterologous nucleotides that do not naturally occur contiguously with the polynucleotides. As used herein the term “heterologous” refers to a combination of elements that are not naturally occurring or that are obtained from different sources.

Polynucleotides can be isolated. An isolated polynucleotide is a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. Polynucleotides can encode full-length polypeptides, polypeptide fragments, and variant or fusion polypeptides.

Degenerate polynucleotide sequences encoding polypeptides described herein, as well as homologous nucleotide sequences that are at least about 80, or about 85, 90, 91, 92, 93, 94, 95, 96, 97, 98, or 99% identical to polynucleotides described herein and the complements thereof are also polynucleotides. Degenerate nucleotide sequences are polynucleotides that encode a polypeptide described herein or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides also are polynucleotides.

Polynucleotides can be obtained from nucleic acid sequences present in, for example, Mycoplasma sp. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides can comprise non-coding sequences or coding sequences for naturally occurring polypeptides or can encode altered sequences that do not occur in nature.

Unless otherwise indicated, the term polynucleotide or gene includes reference to the specified sequence as well as the complementary sequence thereof.

The expression products of genes or polynucleotides are often proteins, or polypeptides, but in non-protein coding genes such as IRNA genes or tRNA genes, the product is a functional RNA. The process of gene expression is used by all known life forms. i.e., eukaryotes (including multicellular organisms), prokaryotes (bacteria and archaea), and viruses, to generate the macromolecular machinery for life. Several steps in the gene expression process can be modulated, including the transcription, up-regulation, RNA splicing, translation, and post-translational modification of a protein.

### Labels

One or more polypeptides described herein (including detector polypeptides) can be conjugated to one or more labels or tags. A label or tag can be coupled directly or indirectly to the desired component of the assay (e.g., an antibody, polypeptide, or support).

In an aspect, a polypeptide can further comprise one or more scaffolds, carriers and/or linkers such as streptavidin (SA) and avidin and derivatives thereof, biotin, immunoglobulins, antibodies (monoclonal, polyclonal, and recombinant), antibody fragments and derivatives thereof, leucine zipper domain of AP-1 (jun and fos), hexa-his (SEQ ID NO: 20) (metal chelate moiety), hexa-hat GST (glutathione S-tranferase) glutathione affinity, calmodulin-binding peptide (CBP), strep-tag, cellulose binding domain, maltose binding protein, S-peptide tag, chitin binding tag, immunoreactive epitopes, epitope tags, E2Tag, HA epitope tag, Myc epitope, FLAG epitope, AU1 and AU5 epitopes, Glu-Glu epitope, KT3 epitope, IRS epitope, Btag epitope, protein kinase-C epitope, VSV epitope, lectins that mediate binding to a diversity of compounds, including carbohydrates, lipids and proteins (e.g. Con A (Canavalia ensiformis) or WGA (wheat germ agglutinin)) and tetranectin or Protein A or G (antibody affinity).

A multimer can comprise a plurality of identical or different polypeptides linked by a linking moiety.

A polypeptide can comprise more than one label, such as two or more labels, wherein said two or more labels are identical or different.

A polypeptide can comprise one or more fluorophore labels, such as fluorescein isothiocyanate, rhodamine, phycoerythrin, phycocyanin, allophycocyanin, o-phthaldehyde, fluorescamine; 2-(4′-maleimidylanilino)naphthalene-6-sulfonic acid, sodium salt; 5-((((2-iodoacetyl)amino)ethyl)amino) naphthalene-1-sulfonic acid; Pyrene-1-butanoic acid; AlexaFluor 350 (7-amino-6-sulfonic acid-4-methyl coumarin-3-acetic acid; AMCA (7-amino-4-methyl coumarin-3-acetic acid); 7-hydroxy-4-methyl coumarin-3-acetic acid; Marina Blue (6,8-difluoro-7-hydroxy-4-methyl coumarin-3-acetic acid); 7-dimethylamino-coumarin-4-acetic acid; Fluorescamin-N-butyl amine adduct; 7-hydroxy-coumarine-3-carboxylic acid; CascadeBlue (pyrene-trisulphonic acid acetyl azide; Cascade Yellow; Pacific Blue (6,8 difluoro-7-hydroxy coumarin-3-carboxylic acid; 7-diethylamino-coumarin-3-carboxylic acid; N-(((4-azidobenzoyl)amino)ethyl)-4-amino-3,6-disulfo-1,8-naphthalimide, dipotassium salt; Alexa Fluor 430; 3-perylenedodecanoic acid; 8-hydroxypyrene-1,3,6-trisulfonic acid, trisodium salt; 12-(N-(7-nitrobenz-2-oxa-1,3-diazol-4-yl)amino)dodecanoic acid; N,N′-dimethyl-N-(iodoacetyl)-N′-(7-nitrobenz-2-oxa-1,3-diazol-4-yl)ethyle-nediamine; Oregon Green 488 (difluoro carboxy fluorescein); 5-iodoacetamidofluorescein; propidium iodide-DNA adduct; Carboxy fluorescein or combinations thereof.

In an aspect, a polypeptide can comprise a fluorescent label, such as a simple fluorescent label, such as Fluor dyes, Pacific Blue®, Pacific Orange®, Cascade Yellow®; AlexaFluor®, AF405, AF488, AF500, AF514, AF532, AF546, AF555, AF568, AF594, AF610, AF633, AF635, AF647, AF680, AF700, AF710, AF750, AF800; Quantum Dot based dyes, QDot® Nanocrystals (Invitrogen, Molecular Probes), Qdot®525, Qdot®565, Qdot@585, Qdot@605, Qdot@655, Qdot®705, Qdot®800; DyLight® Dyes (Pierce) (DL); DL549, DL649, DL680, DL800; Fluorescein (Flu) or any derivate of that, such as FITC; Cy-Dyes, Cy2, Cy3, Cy3.5, Cy5, Cy5.5, Cy7; fluorescent proteins, RPE, PerCp, APC, Green fluorescent proteins; GFP and GFP derivated mutant proteins; BFP, CFP, YFP, DsRed, T1, Dimer2, mRFP1, MBanana, mOrange, dTomato, tdTomato, mTangerine, mStrawberry, mCherry; Tandem dyes, RPE-Cy5, RPE-Cy5.5, RPE-Cy7, RPE-AlexaFluor® tandem conjugates; RPE-Alexa610, RPE-TxRed, APC-Aleca600, APC-Alexa610, APC-Alexa750, APC-Cy5, APC-Cy5.5; multi fluorochrome assemblies, multiple fluorochromes attached to a polymer molecule, such as a peptide/protein, Dextrane, polysaccharide, any combination of the fluorescent dyes involving in generation of FRET (Fluorescence resonance energy transfer) based techniques; ionophors; ion chelating fluorescent props, props that change wavelength when binding a specific ion, such as Calcium, props that change intensity when binding to a specific ion, such as calcium, or combinations thereof.

In an aspect, a polypeptide can comprise one or more labels that are capable of absorption of light, such as a chromophore or a dye. In an aspect a polypeptide can comprise one or more labels that is capable of emission of light after excitation, such as one or more fluorochromes, such as AlexaFluor®(AF) family, which include AF®350, AF405, AF430, AF488, AF500, AF514, AF532, AF546, AF555, AF568, AF594, AF610, AF633, AF635, AF647, AF680, AF700, AF710, AF750 and AF800; selected from the Quantum Dot (Qdot®) based dye family (Qdot525®, Qdot@565, Qdot®585, Qdot®605, Qdot®655, Qdot®705, Qdot@800), the DyLight® Dyes (DL) family (e.g., DL549, DL649, DL680, DL800) the family of small fluorescing dyes (e.g., FITC, Pacific Blue®, Pacific Orange®, Cascade Yellow®, Marina Blue®, DSred, Dsred-2, 7-AAD, TO-Pro-3; the family of Cy-Dyes (e.g., Cy2, Cy3, Cy3.5, Cy5, Cy5.5, Cy7), Phycobili proteins (such as R-Phycoerythrin (RPE), PerCP, Allophycocyanin (APC), B-Phycoerythrin, C-Phycocyanin); fluorescent proteins (such as (E)GFP and GFP ((enhanced) green fluorescent protein) derived mutant proteins; BFP, CFP, YFP, DsRed, T1, Dimer2, mRFP1, MBanana, mOrange, dTomato, tdTomato, mTangerine; Tandem dyes with RPE (e.g., RPE-Cy5, RPE-Cy5.5, RPE-Cy7, RPE-AlexaFluor® tandem conjugates; RPE-Alexa610, RPE-TxRed; tandem dyes with APC (such as APC-Aleca600, APC-Alexa610, APC-Alexa750, APC-Cy5, APC-Cy5.5), and calcium dyes (e.g., Indo-1-Ca2+, Indo-2-Ca2+).

In an aspect, a polypeptide can comprise one or more labels is capable of reflection of light, such as gold, plastic, glass, polystyrene and/or pollen.

In an aspect, a polypeptide can comprise one or more chemiluminescent labels such as luminol, isoluminol, theromatic acridinium ester, imidazole, acridinium salt, or oxalate ester.

In an aspect, a polypeptide can comprise a bioluminescent label (e.g., luciferin, luciferase or aequorin).

In an aspect, a polypeptide can comprise a radioactive label (e.g., such a radionuclide, an isotope, a label comprising a rays, a label comprising p rays or comprising gamma rays).

In an aspect, a polypeptide can comprise an enzyme polypeptide (e.g., an enzyme that can catalyze a reaction between chemicals in the area of the labeling molecules, an enzyme or producing a light signal (chemi-luminescence), the enzyme catalyze a reaction between chemicals in the near environment of the labeling molecules, resulting in precipitation of chromophor dyes, or the enzyme catalyze a reaction between chemicals in the near environment of the labeling molecules, resulting in precipitates that can be detected by an additional layer of detection molecules.

In an aspect, a polypeptide can comprise an enzyme label such as peroxidases, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase, horseradish peroxidase, alkaline phosphatase.

In an aspect, a polypeptide can comprise an ionophore or chelating chemical compound, a lanthanide (e.g., a lanthanide that comprises fluorescence, a lanthanide that comprises phosphorescence, or a paramagnetic lanthanide), a DNA fluorescing stain (e.g., propidium iodide, Hoechst stain, DAPI, AMC, DraQ5.TM. and Acridine orange), a nucleic acid label (e.g., DNA label, an RNA label, or an artificial nucleic acid label and/or a nucleic acid comprising one or more of a) barcode region, b) 5′ first primer region (forward) c) 3′ second primer region (reverse), d) random nucleotide region, e) connector molecule f) stability-increasing components g) short nucleotide linkers in between any of the above-mentioned components h) adaptors for sequencing or i) annealing region).

Detection of labels or tags can be done using many different methods. For example, a radioactive label can be detected using a scintillation counter, photographic film as in autoradiography, or storage phosphor imaging. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Simple colorimetric labels can be detected by observing the color associated with the label. When pairs of fluorophores are used in an assay, they may have distinct emission patterns (wavelengths) so that they can be easily distinguished.

While components described herein (e.g., polypeptides, antibodies and specific binding fragments thereof) can comprise a label, a label is not necessary for detection of polypeptide/antibody complexes since many options exist for label-free detection including, for example surface plasmon resonance, bio-layer interferometry, and grating-coupled interferometry detection assays.

### Supports

In an embodiment, a component of an assay (e.g., a polypeptide or antibody or specific binding fragment thereof) can be immobilized to a support. A support is any material that is appropriate for or that can be modified to be appropriate for attachment of one or more polypeptides, antibodies, or specific binding fragments as described herein. Examples of supports include glass and modified or functionalized glass, plastics (including acrylics, polystyrene, methylstyrene, polyurethanes, Teflon®, etc.), paramagnetic materials, thoria sol, carbon graphite, titanium oxide, latex or cross-linked dextrans such as Sepharose, cellulose polysaccharides, nylon or nitrocellulose, ceramics, resins, silica or silica-based materials including silicon and modified silicon, carbon metals, inorganic glasses, optical fiber bundles, and a variety of other polymers. In an embodiment a support can be located in a microtiter well plate (e.g., a 96-well, 384-well or 1536-well plate). In an embodiment, a support can be located within a flow cell or flow cell apparatus (e.g., a flow cell on a protein chip). A support can be a solid support.

In an embodiment, a support can be a bead such as a magnetic bead, a magnetic barcoded bead, microsphere, particle, membrane, chip, slide, well, or test tube. Beads include microspheres or particles, which can be small, discrete, non-planar particles in the micrometer or nanometer dimensions. A bead can be spherical or irregular. A bead can be porous. In an embodiment, a support can comprise a patterned surface suitable for immobilization of polypeptides in an ordered pattern (e.g., a protein chip).

In an embodiment, one or more polypeptides as described herein can be immobilized to a support via a linker molecule. One or more polypeptides can be conjugated to a support using any suitable methodology. In an embodiment one or more polypeptides are conjugated to a support using a conjugation reagent, including covalent and non-covalent conjugation reagents. Covalent conjugation reagents can include any chemical or biological reagent that can be used to covalently immobilize a polypeptide on a surface. Covalent conjugation reagents include, for example, a carboxyl-to-amine reactive group such as carbodiimides such as EDC or DCC, an amine reactive group such as N-hydroxysuccinimide (NHS) ester or imidoesters, a sulfhydryl-reactive crosslinker such as maleimides, haloacetyls, or pyridyl disulfides, carbonyl-reactive crosslinker groups such as, hydrazides or alkoxyamines, a photoreactive crosslinker such as aryl azides or dizirines, or a chemoselective ligation group such as a Staudinger reaction pair. Non-covalent immobilization reagents can include any chemical or biological reagent that can be used to immobilize a polypeptide non-covalently on a surface, such as affinity tags such as biotin or capture reagents such as streptavidin or anti-tag antibodies, such as anti-His6 (“HHHHHH” disclosed as SEQ ID NO: 20) or anti-Myc antibodies.

### Methods of Detection and Diagnosis

In an embodiment, an assay method can comprise contacting a test sample with one or more polypeptides under conditions suitable for formation of complexes between polypeptides and antibodies or specific binding fragments thereof. The one or more polypeptides can comprise, for example, about 70%, 75%, 80%, 85%, 90%, 95%, 96%, 97%, 98%, 99% sequence identity to SEQ ID NO: 1-12, fragments, or variants of these polypeptides as described herein. Complexes of polypeptides and antibodies or specific binding fragments thereof can be detected. In an embodiment, complexes of polypeptides and antibodies or specific binding fragments can also comprise one or more labels or tags. If complexes are detected, then the sample contains anti-Mycoplasma haemofelis or anti-Mycoplasma haemocanis antibodies.

In an embodiment, a method comprises diagnosing a disease caused by Mycoplasma haemofelis or Mycoplasma haemocanis in a subject. A test sample can be contacted with one or more polypeptides comprising about 70%, 75%, 80%, 85%, 90%, 95%, 96%, 97%, 98%, 99% sequence identity to SEQ ID NO:1-12, fragments, or variants as described herein. Complexes of anti-Mycoplasma haemofelis or anti-Mycoplasma haemocanis antibodies or specific binding fragments thereof present in the sample and the one or more polypeptides are then detected. The subject can have been infected with Mycoplasma haemofelis or anti-Mycoplasma haemocanis for less than about 7, 8, 9, 10, 11, 12, 13, 14, 15, 16, 17, 18, 19, 20, 21, 28, or 35 days. That is, detection (using any method described herein) can occur at about 7, 8, 9, 10, 11, 12, 13, 14, 15, 16, 17, 18, 19, 20, 21, 28, or 35 days after infection. The amount of anti-Mycoplasma haemofelis antibodies, or anti-Mycoplasma haemocanis antibodies or specific binding fragments thereof in the sample can be determined. The amount of the complexes in the sample can be compared to a control sample or control standard, wherein elevated levels of the complexes as compared to the control sample or control standard is an indication of a disease caused by Mycoplasma haemofelis or Mycoplasma haemocanis. Where the complexes are detected a treatment can be administered to the subject. A subject can be human or a non-human mammal, such as a dog, horse, cow, or cat. A test sample can be, for example, blood, plasma, serum, lymph fluid, or any other body fluid sample. In some aspects a test sample is removed from the subject or patient and methods of detection and diagnosis are completed ex vivo or in vitro.

In an embodiment, specific binding between one or more polypeptides described herein and one or more anti-Mycoplasma haemofelis antibodies, anti-Mycoplasma haemocanis antibodies or specific binding fragments thereof are detected using a secondary antibody or a specific binding fragment thereof. In an example, a secondary antibody can be used to detect the specific binding. For example, a secondary antibody or specific binding fragment thereof is capable of binding the anti-Mycoplasma haemofelis antibodies, the anti-Mycoplasma haemocanis antibodies, or specific binding fragments thereof. In an embodiment a secondary antibody is an anti-species antibody such as a rabbit anti-feline or rabbit anti-canine antibody. In an embodiment, the secondary antibody is covalently or non-covalently bound to a label or tag that can be used for detection of polypeptide/anti-Mycoplasma antibody/secondary antibody complex. In one example, a secondary antibody can be conjugated to biotin or other label or tag. A streptavidin conjugate (in the case of a biotin label), such as streptavidin fluorochrome conjugate, can be used to detect the polypeptide/anti-Mycoplasma antibody/secondary antibody complex.

In an embodiment, specific binding between one or more polypeptides described herein and one or more anti-Mycoplasma haemofelis antibodies anti-Mycoplasma haemocanis antibodies or specific binding fragments thereof are detected using one or more detector polypeptides that specifically bind anti-Mycoplasma haemofelis antibodies, anti-Mycoplasma haemocanis antibodies, or specific binding fragments thereof. In an example, the one or more detector polypeptides comprise about 70%, 75%, 80%, 85%, 90%, 95%, 96%, 97%, 98%, 99% sequence identity to a polypeptide as set forth in SEQ ID NOs: 1-12, fragments thereof, or variants as described herein. In an embodiment, the one or more detector polypeptides are covalently or non-covalently bound to a label or tag that can be used for detection of polypeptide/anti-Mycoplasma antibody/detector polypeptide complex. In one example, a detector polypeptide can be conjugated to a label or tag such as biotin. A streptavidin conjugate (in the case of a biotin label), such as streptavidin fluorochrome conjugate can be used to detect the polypeptide/anti-Mycoplasma antibody/detector polypeptide complex.

In an embodiment, one or more anti-Mycoplasma haemofelis antibodies, anti-Mycoplasma haemocanis antibodies, or specific binding fragments thereof can be immobilized to a support. In an aspect, these antibodies are polyclonal antibodies. One or more of the polypeptides described herein can specifically bind the immobilized antibodies. A sample can be added to the assay and any anti-Mycoplasma haemofelis antibodies, anti-Mycoplasma haemocanis antibodies or specific binding fragments thereof will specifically bind to the one or more polypeptides. The antibodies from the sample can be detected with labeled secondary antibodies, for example, anti-feline or anti-canine secondary antibodies.

The detection of specific binding between one or more polypeptides described herein and one or more antibodies or specific binding fragments thereof can be completed using any suitable method, for example, an immunoassay such as a competitive immunoassay, a sandwich immunoassay, an enzyme-linked immunosorbent assay (ELISA), an immunohistochemical assay, a turbidimetric immunoassay, a particle-enhanced turbidimetric immunoassay, a radioimmunoassay (RIA), a fluorescent immunosorbent assay (FIA), a multiplex immunoassay, a protein/peptide array immunoassay, a solid phase radioimmunoassay (SPRIA), an indirect immunofluorescence assay (IIF), a chemiluminescent immunoassay (CIA), a particle based multianalyte test (PMAT), a dot blot assay, or a western blot assay. Other assay methods that can be used to detect antibodies and/or antibody/polypeptide complexes include, for example surface plasmon resonance (SPR), isothermal titration calorimetry (ITC), microscale thermophoresis (MST), biolayer interferometry, or grating-coupled interferometry.

Antibodies described herein can be a polyclonal antibody, a monoclonal antibody, a single chain antibody (scFv), or a specific binding fragment of an antibody. Antibody specific binding fragments are one or more portions of an intact antibody that comprises the antigen binding site or variable region of an intact antibody, where the portion is free of the constant heavy chain domains of the Fc region of the antibody. Examples of antibody fragments that bind to antigens include Fab, Fab′, Fab′-SH, F(ab′)2 and Fv fragments. An antibody described herein can be any class of antibody that includes, for example, IgG, IgM, IgA, IgD and IgE. In an aspect one or more of the polypeptides described herein can be used to generate polyclonal antibodies using rabbit or any other mammals. In one technique, the polypeptide of the disclosure is introduced into a host animal, such as into rabbit, mouse, rat, guinea pig, goat, pig, cow, sheep, donkey, dog, cat, chicken, or horse, for example. An enhanced immune response may be elicited in the host animal by associating the polypeptide with a carrier and/or by exposing the host to an adjuvant, but it is to be understood that the present disclosure does not require that the polypeptide be associated with a carrier or that the host be exposed to the adjuvant. An exemplary carrier that may be used for this purpose is bovine serum albumin, bovine thyroglobulin, and soybean trypsin inhibitor. Exemplary adjuvants include Freund's complete or incomplete adjuvant and MDL-TDM adjuvant. Regardless of whether the polypeptide is associated with such a carrier or whether the host is exposed to an adjuvant, booster immunizations optionally may be made with the host animal being bled one or more times thereafter. Polyclonal antibodies that specifically bind the polypeptide may then be purified from antisera obtained from the bleed or bleeds. Such purification may be achieved, for example, by employing affinity chromatography techniques that involve associating the polypeptide to a solid support. Such affinity chromatography techniques are well known by the skilled artisan.

These polypeptides can be used in the assays as described herein. A polyclonal antibody population can be prepared by administering an antigen (e.g., one or more polypeptides described herein) or an antigenic fragment thereof, to a mammal (e.g., a rabbit) and purifying a population of polyclonal antibodies from serum or plasma of the mammal. These polyclonal antibodies can specifically bind one or more of the polypeptides described herein.

“Specifically binds” or “specific for” means that a first antigen, e.g., a polypeptide as shown in SEQ ID NOs:1-12, fragments thereof, or variants as described herein, recognizes and binds to an anti-Mycoplasma haemofelis antibody, an anti-Mycoplasma haemocanis antibody, or specific binding fragments thereof with greater affinity than other non-specific molecules. A non-specific molecule is an antigen that shares no common epitope with the first antigen. In an embodiment, a non-specific molecule is not a Mycoplasma haemofelis polypeptide and is not related to Mycoplasma haemofelis. In an embodiment, a non-specific molecule is not a Mycoplasma haemocanis polypeptide and is not related to Mycoplasma haemocanis. In an embodiment, a non-specific molecule is not derived from a Mycoplasma organism. For example, an antibody raised against a first antigen (e.g., Mycoplasma haemofelis) to which it binds more efficiently than to a non-specific antigen can be described as specifically binding to the first antigen. A polypeptide specifically binds to an anti-Mycoplasma haemofelis antibody, anti-Mycoplasma haemocanis antibody, or specific binding fragment when it binds with a binding affinity (K_{D}) of 10-6 M or less. In an embodiment, a polypeptide specifically binds to an anti-Mycoplasma haemofelis antibody, anti-Mycoplasma haemocanis antibody, or specific binding fragment when it binds with an affinity (K_{D}) of 2×10⁻⁶ M or less. In an embodiment, a polypeptide specifically binds to an anti-Mycoplasma haemofelis antibody, anti-Mycoplasma haemocanis antibody, or specific binding fragment when it binds with an affinity (K_{D}) of at least 10-6 M, 10-7 M, 10-8 M, 10-9 M or less. In certain embodiments, the affinity is measured by surface plasmon resonance or KinExA assay. In an embodiment, the methods specifically detect Mycoplasma haemofelis and do not detect other species of Mycoplasma. In an embodiment, the methods specifically detect Mycoplasma haemocanis and do not detect other species of Mycoplasma. In an embodiment, the methods specifically detect Mycoplasma haemofelis and Mycoplasma haemocanis.

### Kits

A kit can comprise one or more of the polypeptides described herein. A kit can comprise one or more anti-Mycoplasma haemofelis antibodies, anti-Mycoplasma haemocanis antibodies, or specific binding fragments thereof. A kit can comprise anti-species secondary antibodies like anti-feline species antibodies or anti-canine species antibodies. Any of these components can be labeled and/or immobilized to a support.

For example, a label of the kit can include a fluorophore, an enzyme, a chemiluminescent moiety, a radioactive moiety, an organic dye, a small molecule, a polypeptide or functional fragment thereof. In some embodiments, a label of the kit includes phycoerythrin (PE). In some embodiments, a label of the kit includes fluorescein isothiocyanate. In some embodiments, a label is conjugated to a secondary antibody or detector polypeptide.

A kit can include a positive control or standard. In some embodiments, a positive control or standard can be a sample containing a detectable amount of anti-Mycoplasma haemofelis antibodies and/or anti-Mycoplasma haemocanis antibodies. In an embodiment, a positive control or standard can be obtained from a diseased subject who has anti-Mycoplasma haemofelis antibodies or anti-Mycoplasma haemocanis antibodies. In an embodiment, a positive control or standard can comprise anti-Mycoplasma haemofelis antibodies or anti-Mycoplasma haemocanis antibodies synthesized in vitro or otherwise obtained. In an embodiment, a kit can include a negative control or standard. A negative control or standard can be a sample containing no detectable amount of anti-Mycoplasma haemofelis antibodies or anti-Mycoplasma haemocanis antibodies. In some embodiments, a negative control or standard can be obtained from a healthy control individual or can be synthesized in vitro. For example, a negative control can include water or buffer.

In some embodiments a kit can include a standard curve for determining an amount of anti-Mycoplasma haemofelis antibodies, Mycoplasma haemofelis polypeptides, anti-Mycoplasma haemocanis antibodies, or Mycoplasma haemocanis polypeptides in a sample. Anti-Mycoplasma haemofelis antibodies, Mycoplasma haemofelis polypeptides, anti-Mycoplasma haemocanis antibodies, or Mycoplasma haemocanis polypeptides can be used to create a standard curve for an assay. A standard curve can be used to determine the concentration of anti-Mycoplasma haemofelis antibodies, Mycoplasma haemofelis polypeptides, anti-Mycoplasma haemocanis antibodies, or Mycoplasma haemocanis polypeptides in a sample. A standard curve is obtained by relating a measured quantity to the concentration of the anti-Mycoplasma haemofelis antibodies, Mycoplasma haemofelis polypeptides, anti-Mycoplasma haemocanis antibodies, or Mycoplasma haemocanis polypeptides in “known” samples, i.e., standards of known concentration. These standards provide a reference to determine unknown concentrations of anti-Mycoplasma haemofelis antibodies, Mycoplasma haemofelis polypeptides, anti-Mycoplasma haemocanis antibodies, or Mycoplasma haemocanis polypeptides in a sample. The amounts of standards can span the whole range of concentrations expected to be found in the “unknown” or “test” sample concentration.

A kit can further include one or more assay reagents that facilitate binding of the one or more polypeptides to anti-Mycoplasma haemofelis antibodies, anti-Mycoplasma haemocanis antibodies or specific binding fragments thereof. The assays reagents can be substances, mixtures, materials, or components that are useful to carry out an intended purpose of the kit. Reagents can include, for example, a conjugation reagent, a buffer, standard, positive control, label, a sample collection device, instructions and the like.

A kit can include one or more buffers, such as a wash buffer. A wash buffer can include, for example, tris(hydroxymethyl)aminomethane (Tris)-based buffers like Tris-buffered saline (TBS) or phosphate buffers like phosphate-buffered saline (PBS). Wash buffers can be composed of, for example, detergents, such as ionic or non-ionic detergents. In some embodiments, a wash buffer can be a PBS buffer at about pH 7.0, 7.2, 7.4, 7.6, 7.8 including Tween™20 (polysorbate) at about 0.001, 0.01, 0.02, 0.03, 0.04, 0.05, 0.06, 0.07, 0.08% or more.

A kit can include a dilution buffer. Dilution buffers include, for example, a carrier protein such as bovine serum albumin (BSA) and a detergent such as Tween®20 (polysorbate).

A kit can include a detection or assay buffer. A detection or assay buffer can be, for example, a colorimetric detection or assay buffer, a fluorescent detection or assay buffer, or a chemiluminescent detection or assay buffer. Colorimetric detection or assay buffers include, for example, PNPP (p-nitrophenyl phosphate), ABTS (2,2′-azino-bis(3-ethylbenzothiazoline-6-sulphonic acid)) or OPD (o-phenylenediamine). Fluorescent detection or assay buffers include QuantaBlu® or QuantaRed® (Thermo Scientific, Waltham, MA). Chemiluminescent detection or assay buffers can include luminol or luciferin. Detection or assay buffers can also include a trigger such as H₂O₂ and a tracer such as isoluminol-conjugate.

A kit can include a stop solution. Stop solutions of can terminate or delay the further development of the detection reagent and corresponding assay signals. Stop solutions can include, e.g., low-pH buffers (e.g., glycine-buffer, pH 2.0), chaotropic agents (e.g., guanidinium chloride, sodium-dodecylsulfate (SDS)), reducing agents (e.g., dithiothreitol, B-mercaptoethanol), or the like.

A kit provided can include a device for collecting a biological sample, such as collection tubes, columns, swabs, syringes, and needles. A kit can include instructions for using the components of the kit. The instructions can provide details regarding protocols and analytical techniques.

Components of a kit can be in any physical state. For example, one or more of the components can be lyophilized, in aqueous solution, or frozen.

A kit of can be designed for specific assay technologies. For example, a kit can be an immunoassay kit, a competitive immunoassay kit, a sandwich immunoassay kit, an enzyme-linked immunosorbent assay (ELISA) kit, an immunohistochemical assay kit, a turbidimetric immunoassay kit, a particle-enhanced turbidimetric immunoassay kit, a radioimmunoassay (RIA) kit, a fluorescent immunosorbent assay (FIA) kit, a multiplex immunoassay kit, a protein/peptide array immunoassay kit, a solid phase radioimmunoassay (SPRIA) kit, an indirect immunofluorescence assay (IIF) kit, a chemiluminescent immunoassay (CIA) kit, a particle based multianalyte test (PMAT) kit, a dot blot assay kit, or a western blot assay kit, a surface plasmon resonance (SPR) kit, an isothermal titration calorimetry (ITC) kit, a microscale thermophoresis (MST) kit, a biolayer interferometry kit, or grating-coupled interferometry kit. In an embodiment, an ELISA kit can include, for example, a wash buffer, sample diluent, a secondary antibody, a secondary antibody-enzyme conjugate, a detector polypeptide, a labeled detector polypeptide, a detection reagent, and a stop solution. In an embodiment a dot blot kit can include, for example, a wash buffer, sample diluent, a secondary antibody-enzyme conjugate, a detection reagent, and a stop solution. In some embodiments, a chemiluminescent immunoassay kit can include a wash buffer, a sample diluent, a tracer (e.g., isoluminol-conjugate) and a trigger (e.g., H₂O₂). In an embodiment, a multiplex kit can include, for example, a wash buffer, a sample diluent, and a secondary antibody-enzyme conjugate.

### Methods of Treatment

Once detected or diagnosed, Mycoplasma haemofelis or Mycoplasma haemocanis can be treated with, for example, antibiotic drugs, protein or peptide drugs, nucleic acid-based drugs, anti-inflammatory drugs, other drugs, immunomodulatory treatments, alternative therapies, or combinations thereof.

For example, Mycoplasma haemofelis or Mycoplasma haemocanis can be treated with a number of antibiotics, for example, tetracyclines, fluoroquinolones (e.g., Enrofloxacin), doxycycline, furosemide, robenidine, or marbofloxacin. Antibiotic treatment can be about administered at about 5-10 mg/kg for up to 28 days. In an aspect doxycycline can be used at 5-10 mg/kg for up to 28 days. If bacteremia persists or reoccurs, antibiotic treatment can be switched to marbofloxacin (2 mg/kg once daily for 14 days) Other drugs such as robenidine and those listed in U.S. Pat. No. 10,370,341 (incorporated by reference herein) can be used for treatment. Glucocorticoids can also be used to decrease erythrophagocytosis in cases of severe hemolysis because the primary mechanism of damage is immune-mediated targeting of infected host cells. Supportive care such as oxygen and blood transfusions can be used for treatment.

Methods of treatment for Mycoplasma haemofelis or Mycoplasma haemocanis can include contacting a test sample with the one or more polypeptides described herein. Complexes of anti-Mycoplasma haemofelis or anti-Mycoplasma haemocanis or specific binding fragments thereof and the one or more polypeptides can be detected as described herein. The amount of complexes can be compared to a positive control or standard and/or to a negative control or standard. Where the amount of complexes indicates an infection, the subject can be treated with one or more antibiotics or other treatments described herein and/or known to those of skill in the art.

A control or standard can be used for comparison. A control or standard value can be determined prior to running an assay, in parallel, simultaneously, in a multiplex assay or other assay format. A control or standard can be a first level determined by assay in a first sample obtained from a patient. A control or standard can be level or range detected in a population of healthy subjects, Mycoplasma haemofelis or Mycoplasma haemocanis patients or severe Mycoplasma haemofelis or Mycoplasma haemocanis patients, for example.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLE 1

### Discovery of Marker TDX1362

The Mycoplasma haemofelis genome comprises about 80% hypothetical proteins, that is proteins with no predicted function. Potential useful markers were selected by sequence analysis and integrative analysis. The genome of M. haemofelis was compared with non-pathogenic M. haemominutum genome. 13 different proteins unique to M. haemofelis were selected for screening with infected samples. Thirteen promising markers were cloned into expression vectors. The expressed proteins were subjected to immunoblot screening. Several of the expressed proteins were purified and subjected to ELISA analysis for binding to Mycoplasma haemofelis-specific antibodies. The polynucleotide encoding TDX1362 was sequenced (see SEQ ID NO:9). Full length TDX1362 was cloned into pET28a so that the expressed protein included a pET28a 6xHis tag (SEQ ID NO: 20) and a thrombin cleavage site (see SEQ ID NO: 13). To express the recombinant protein, the signal sequence was removed and only the sequence of gene encoding the mature protein was cloned into pET28a vector. The N-terminal region was tagged with His tag and Thrombin site (MGSSHHHHHHSSGLVPRGSH, SEQ ID NO:13). The recombinant protein TDX1362 was expressed in E. coli BL21 using IPTG induction. The expressed recombinant protein migrated as per the predicted molecular weight of 21 kDa. The mature protein is shown in SEQ ID NO: 10.

Recombinant TDX1362 protein was purified using an IMAC column. Briefly, the cells were suspended in PBS pH 7.2 and lysed with a cell disruptor. The lysate was centrifuged for 10,000 g for 30 minutes. The supernatant was carefully removed and subjected to purification with IMAC column. The purified recombinant TDX1362 was checked in SDS-PAGE to confirm its purity. The total yield of the recombinant protein was estimated by OD280. About 30 mgs per liter was recovered. Immunoblot screening against sera of M. haemofelis experimentally infected cats, naturally M. haemofelis infected cats, and negative screening confirmed that the TDX1362 protein was immunoreactive.

An indirect ELISA assay was designed wherein the TDX1362 was immobilized to an Immulon IV plate. Briefly, the plates were coated with recombinant TDX1362 for an overnight period at 4° C. and the plates were washed with PBST and blocked with 2% BSA for 2-3 hours. The plates were washed again and stored at 4° C. for future use. The plates thawed at RT for 3-4 hours were used in the experiment. The serum samples were diluted with PRRS diluent and added to the plates and incubated for an hour at RT. The plates were washed again with PBST and the secondary antibody goat anti-lgG HRP diluted in enzyme conjugate diluent was added and incubated for 30 min at RT. The plates were washed with PBST and the substrate was added and read at 450 nm. Sera from experimentally infected M. haemofelis cats, sera from samples that were PCR positive for M. haemofelis, samples that were PCR negative for M. haemofelis, and samples from a feline general population were added to the assay plates. Labeled anti-feline IgG was added to the assay plates. The label was then detected.

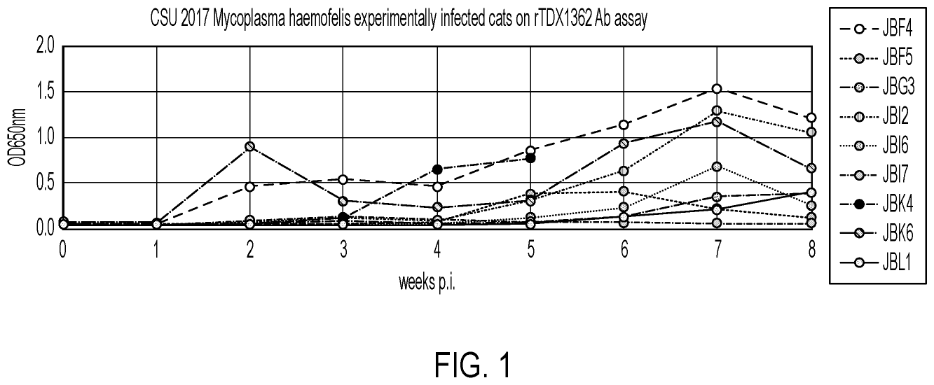

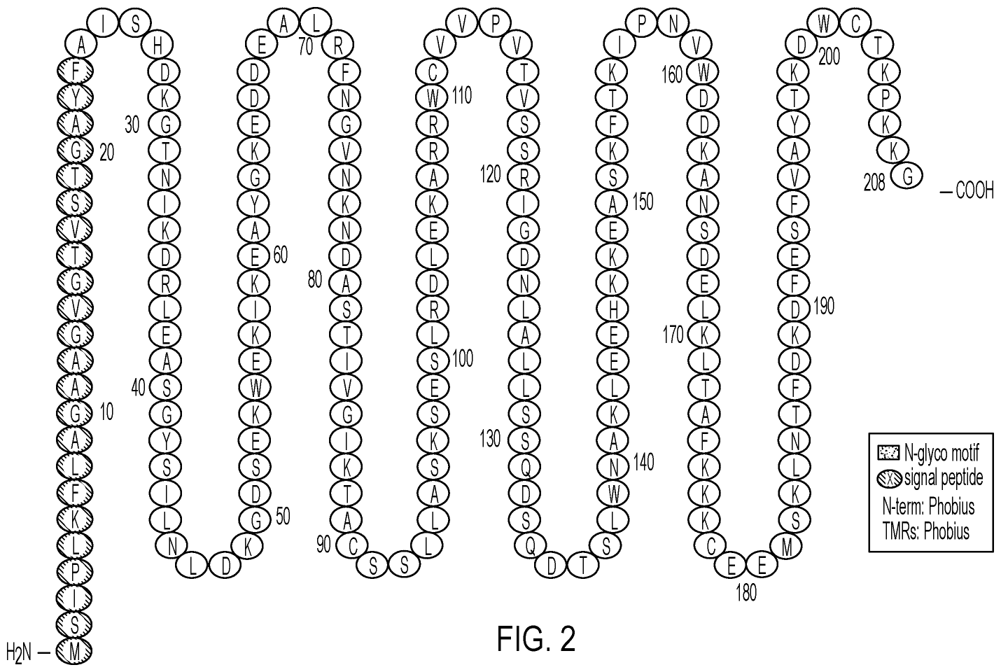

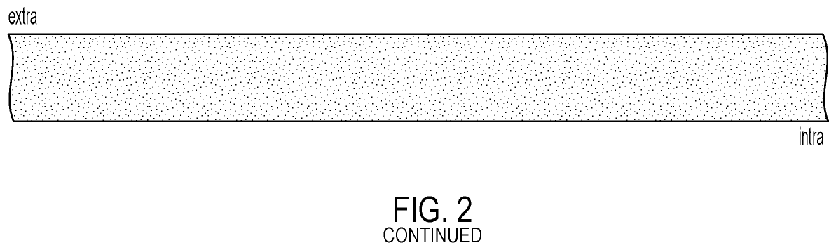

The indirect ELISA assay was used to test for antibody response in M. haemofelis experimentally infected cats over time. The recombinant protein was coated on an Immulon IV plate for an overnight period at 4° C., and the plate was blocked with blocking solution (1% BSA in 100 mM Tris pH 7.5). The plates were washed with 5×PBST, and the serum samples from experimentally infected cats were diluted with PRRS diluent and added to the plates, and kept it for an hour at RT. The plates were washed again with 5×PBST, and the secondary antibody (Anti-Cat IgG H+L) was added to the plates and incubated for 30 minutes. The plates were washed, and the substrate was added and read at 450 nm. The indirect ELISA assay was able to detect anti-M. haemofelis antibodies in the sera samples as early as about 1.5 weeks. See FIG. 1.

### EXAMPLE 2

TDX1362 was subjected to epitope mapping. 15-mer peptides covering TDX1362 with 10 amino acid overlaps were tested as follows:

A1
SEQ ID NO: 22
01 LCBiot-MAISHDKGTNIKDRL-amide 15
A2
SEQ ID NO: 23
02 LCBiot-DKGTNIKDRLEASGY-amide 15
A3
SEQ ID NO: 24
03 LCBiot-IKDRLEASGYSILNL-amide 15
A4
SEQ ID NO: 25
04 LCBiot-EASGYSILNLDKGDS-amide 15
A5
SEQ ID NO: 26
05 LCBiot-SILNLDKGDSEKWEK-amide 15
A6
SEQ ID NO: 27
06 LCBiot-DKGDSEKWEKIKEAY-amide 15
A7
SEQ ID NO: 28
07 LCBiot-EKWEKIKEAYGKEDD-amide 15
A8
SEQ ID NO: 29
08 LCBiot-IKEAYGKEDDEALRF-amide 15
A9
SEQ ID NO: 30
09 LCBiot-GKEDDEALRFNGVNK-amide 15
A10
SEQ ID NO: 31
10 LCBiot-EALRFNGVNKNDAST-amide 15
A11
SEQ ID NO: 32
11 LCBiot-NGVNKNDASTIVGIK-amide 15
A12
SEQ ID NO: 33
12 LCBiot-NDASTIVGIKTACSS-amide 15
B1
SEQ ID NO: 34
13 LCBiot-IVGIKTACSSLLASK-amide 15
B2
SEQ ID NO: 35
14 LCBiot-TACSSLLASKSESLR-amide 15
B3
SEQ ID NO: 36
15 LCBiot-LLASKSESLRDLEKA-amide 15
B4
SEQ ID NO: 1765
16 LCBiot-SESLRDLEKARRWCV-amide 15
B5
SEQ ID NO: 37
17 LCBiot-DLEKARRWCVVPVTV-amide 15
B6
SEQ ID NO: 38
18 LCBiot-RRWCVVPVTVSSRIG-amide 15
B7
SEQ ID NO: 39
19 LCBiot-VPVTVSSRIGDNLAL-amide 15
B8
SEQ ID NO: 40
20 LCBiot-SSRIGDNLALLSSQD-amide 15
B9
SEQ ID NO: 41
21 LCBiot-DNLALLSSQDSQDTS-amide 15
B10
SEQ ID NO:42
22 LCBiot-LSSQDSQDTSLWNAK-amide 15
B11
SEQ ID NO: 43
23 LCBiot-SQDTSLWNAKLEEHK-amide 15
B12
SEQ ID NO: 44
24 LCBiot-LWNAKLEEHKKEASK-amide 15
C1
SEQ ID NO: 45
25 LCBiot-LEEHKKEASKFTKIP-amide 15
C2
SEQ ID NO: 46
26 LCBiot-KEASKFTKIPNVWDD-amide 15
C3
SEQ ID NO: 47
27 LCBiot-FTKIPNVWDDKANSD-amide 15
C4
SEQ ID NO: 48
28 LCBiot-NVWDDKANSDELKLT-amide 15
C5
SEQ ID NO: 49
29 LCBiot-KANSDELKLTAFKKK-amide 15
C6
SEQ ID NO: 50
30 LCBiot-ELKLTAFKKKCEEMS-amide 15
C7
SEQ ID NO: 51
31 LCBiot-AFKKKCEEMSKLNTF-amide 15
C8
SEQ ID NO: 52
32 LCBiot-CEEMSKLNTFDKDFE-amide 15
C9
SEQ ID NO: 53
33 LCBiot-KLNTFDKDFESFVAY-amide 15
C10
SEQ ID NO: 54
34 LCBiot-DKDFESFVAYTKDWC-amide 15
C11
SEQ ID NO: 1777
35 LCBiot-SFVAYTKDWCTKPKK-amide 15
C12
SEQ ID NO: 55
36 LCBiot-TKDWCTKPKKG-amide 11
B4:
(SEQ ID NO: 1765)
LCBiot-SESLRDLEKARRWCV-amide 15
B5:
(SEQ ID NO: 37)
LCBiot-DLEKARRWCVVPVTV-amide 15
C10:
(SEQ ID NO: 54)
LCBiot-DKDFESFVAYTKDWC-amide 15
C11:
(SEQ ID NO: 1777)
LCBiot-SFVAYTKDWCTKPKK-amide 15.

The sequence overlap between B4 and B5 indicates that antibody from M. haemofelis infected cats binds within the polypeptide sequence that is shared by B4 and B5.

B4/B5:
(SEQ ID NO: 74)
DLEKARRWCV

The sequence overlap between C10 and C11 indicates that antibody from M. haemofelis infected cats binds within the polypeptide sequence that is shared by C10 and C11.

C10/C11:
(SEQ ID NO: 140)
SFVAYTKDWC

36 peptides (15-mers) with 10 amino acid overlap covering the entire full length protein were synthesized and labeled with biotin at the N-terminus.

Neutravidin plates were coated with 100 ul of peptides diluted in PBS. The plates were incubated for 1 hour at RT, with shaking and then washed 3× with PBS-T. Feline serum samples from cats infected with M. haemofelis were diluted in 1×PBS pH 7.2+2% BSA. Plates were incubated for 1 hour at RT, with shaking and then washed 3X with PBS-T. 100 ul of secondary antibody (Anti cat IgG H+L) was added to each well with a dilution rate of 1:5000 dilution. The plates were incubated for 1 hour at RT, with shaking and then washed 5× with PBS-T The plates were developed with 100 ul TMB substrate. Negative samples (i.e., sera from cats not infected with M. haemofelis) showed no significant reactivity to the peptides. Peptides B4 (SEQ ID NO:5), B5 (SEQ ID NO:37), C10 (SEQ ID NO:54), and C11 (SEQ ID NO:7) showed reactivity with sera from M. haemofelis infected cats in this assay, thus identifying immunodominant regions.

B4, B5, C10, and C11 were selected for testing for immunogenicity in rabbits immunized with recombinant TDX1362 protein. Briefly, 100 ul of peptides diluted in PBS were coated on neutravidin plates. The plates were incubated for 1 hour at RT, with shaking. The plates were washed 3× with PBS-T. Serum samples from the rabbits immunized against TDX1362 were diluted in 1×PBS pH 7.2+2% BSA. The plates were incubated for 1 hour at RT, with shaking. Plates were washed 3× with PBS-T. 100 ul of secondary antibody (Anti Rabbit IgG H+L) was added to each well at a dilution of 1:5000. Plates were incubated for 1 hour at RT, with shaking and then washed 5× with PBS-T. Plates were developed with 100 ul TMB substrate. Negative samples showed no significant reactivity to the peptides. B4 and B5 weakly detected antibodies in sera from rabbits immunized with recombinant TDX1362 protein. C10 and C11 did not detect antibodies in sera from rabbits immunized with recombinant TDX1362 protein..

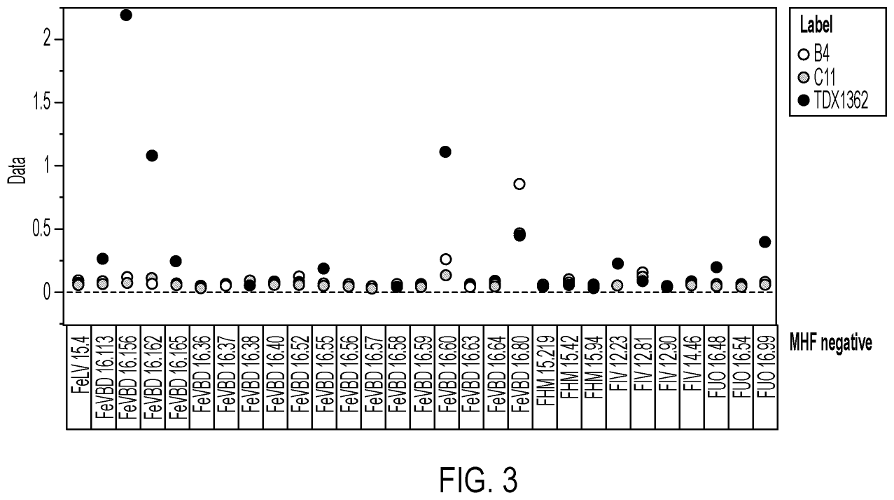

The B4, C11, and the mature TDX1362 polypeptides were tested with M. haemofelis negative sera samples. See FIG. 3. TDX1362 had a specificity of 21/29; C11 had a specificity of 26/29, and B4 had a specificity of 25/29 in the ELISA assay.

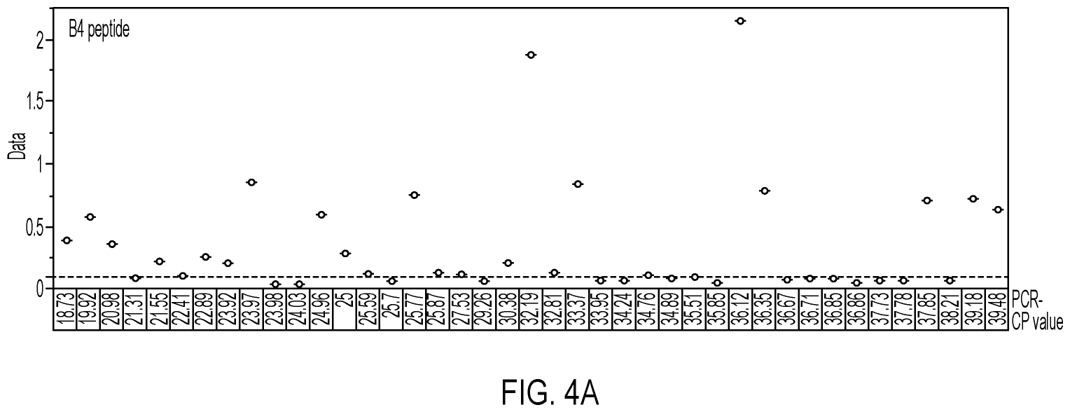

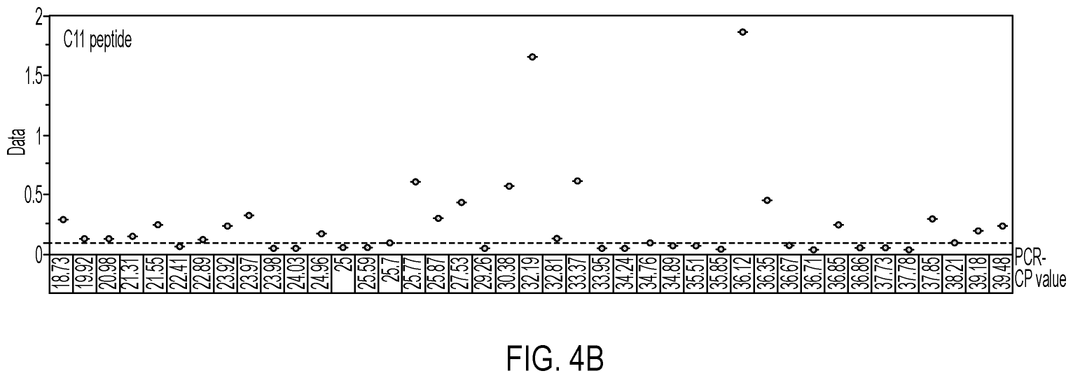

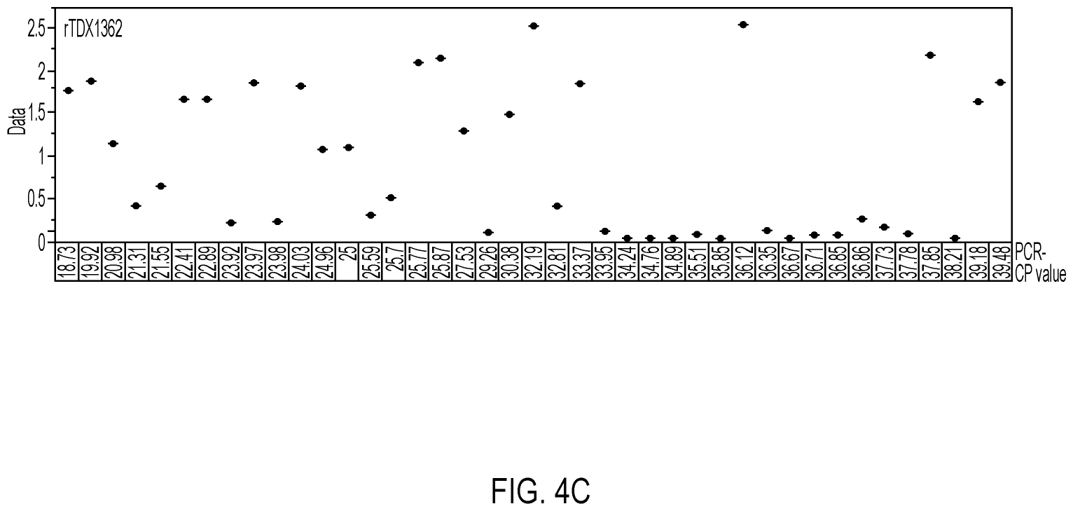

The B4, C11, and the mature TDX1362 polypeptides were tested with M. haemofelis positive sera samples. See FIG. 4A-C. TDX1362 had a sensitivity of 29/41; C11 had a sensitivity of 22/41, and B4 had a sensitivity of 25/41 in the ELISA assay.

Polyclonal antibodies against the recombinant TDX1362 protein were generated in rabbits using a standard protocol (SDIX, LLC, Newark, DE, USA). Briefly, preimmune sera were collected before the immunization of the rabbits. The animals were subcutaneously immunized with 200 ug of immunogen with Freund's complete adjuvant, and then a booster dose was provided on day 21, day 35, and day 50. Bloods were collected on day 45, day 60, and day 70. Serum samples derived from the bloods were evaluated using an ELISA assay with recombinant antigen-coated plates to determine the antibody titer against the immunogen. An antigen capture assay was designed using biotinylated rabbit polyclonal anti-M. haemofelis antibodies. The biotinylated rabbit polyclonal anti-M. haemofelis antibodies were immobilized to a neutravidin plate. Sera samples were added to the plates such that any M. haemofelis antigens present in the samples were able to specifically bind to the biotinylated rabbit polyclonal anti-M. haemofelis antibodies. Assays were also conducted with test samples of M. haemofelis antigens. Horseradish peroxidase labeled rabbit polyclonal anti-M. haemofelis antibodies were used to detect the binding reaction.

An evaluation of the antigen capture assay was conducted with a positive control of recombinant mature TDX1362 at a concentration of 1 μg/ml, 2 μg/ml, or 4 μg/ml, and a negative control of recombinant control TDX926 at a concentration of 1 μg/ml, 2 μg/ml, or 4 μg/ml. The concentration of M. haemofelis antigen was 1, 0.5, 0.25, 0.125, 0.065, 0.0313, 0.0156, 0.0078, 0.0039, 0.002, 0.0001, and 0.0005 μg/ml. Briefly, the purified rabbit IgG against rTDX1362 and rTDX926 (negative control) were coated on to plates (5 μg/ml). The recombinant protein TDX1362 was serially diluted from 1 μg/ml to 0.005 μg/ml concentration and incubated on to lgG coated plates for 30 minutes and the plate was washed 3× with PBST to remove non-specific binding. The purified pAb HRP conjugate against rTDX1362 and rTDX926 were added and kept for an hour and the plate was washed again and the substrate was added, and the plate read at 450 nm. The results are shown in Table 2 below. 2 μg/ml of conjugate was determined the optimal concentration based on these assays.

TABLE 2

| | Positive control: rTDX1362 | | | Negative control: rTDX926 | | |
|---|---|---|---|---|---|---|
| Conc. of | Horseradish peroxidase pAb | | | Horseradish peroxidase pAb | | |
| antigen | conjugate concentration | | | conjugate concentration | | |
| (ug/mL) | 1 ug/ml | 2 ug/ml | 4 ug/ml | 1 ug/ml | 2 ug/ml | 4 ug/ml |
| 1 | 0.93 | 1.0135 | 1.4875 | 0.037 | 0.072 | 0.2 |
| 0.5 | 0.9565 | 1.072 | 1.54 | 0.033 | 0.033 | 0.0485 |
| 0.25 | 0.941 | 1.2045 | 1.5425 | 0.035 | 0.0355 | 0.035 |
| 0.125 | 0.9495 | 1.167 | 1.4845 | 0.0395 | 0.037 | 0.0415 |
| 0.0625 | 0.875 | 1.168 | 1.2575 | 0.039 | 0.036 | 0.0845 |
| 0.0313 | 0.786 | 1.024 | 1.1835 | 0.0465 | 0.041 | 0.0575 |
| 0.0156 | 0.6375 | 0.9045 | 0.9795 | 0.0375 | 0.04 | 0.045 |
| 0.0078 | 0.379 | 0.634 | 0.9115 | 0.0375 | 0.088 | 0.042 |
| 0.0039 | 0.161 | 0.3565 | 0.7135 | 0.0415 | 0.043 | 0.0455 |
| 0.002 | 0.0785 | 0.165 | 0.509 | 0.04 | 0.044 | 0.08 |
| 0.001 | 0.052 | 0.075 | 0.203 | 0.0385 | 0.0435 | 0.042 |
| 0.0005 | 0.051 | 0.0555 | 0.0965 | 0.0835 | 0.0445 | 0.047 |

Samples from PCR positive and PCR negative sera from cats were also tested. The results are shown in Table 3 below.

TABLE 3

| | Conjugate | |
|---|---|---|
| Type | 1 ug/mL | 2 ug/mL |
| PCR pos-1 | 0.95 | 1.246 |
| PCR pos-2 | 0.096 | 0.116 |
| Neg-1 | 0.042 | 0.039 |
| Neg-2 | 0.06 | 0.072 |

The results show that 2 μg/ml of conjugate is suitable for use in detection assays.

### EXAMPLE 3

An alanine scanning analysis of polypeptides B4 and C11 was completed.

B4:
(SEQ ID NO: 1765)
LCBiot-SESLRDLEKARRWCV-amide 15
C11:
(SEQ ID NO: 1777)
LCBiot-SFVAYTKDWCTKPKK-amide 15

The alanine substitution mutants of B4 are shown below

| NO: | Name | IDR mutant |
|---|---|---|
| 1 | B4 M1 | [symbol] ESLRDLEKARRWCV |
| 2 | B4 M2 | S [symbol] SLRDLEKARRWCV |
| 3 | B4 M3 | SE [symbol] LRDLEKARRWCV |
| 4 | B4 M4 | SES [symbol] RDLEKARRWCV |
| 5 | B4 M5 | SESL [symbol] DLEKARRWCV |
| 6 | B4 M6 | SESLR [symbol] LEKARRWCV |
| 7 | B4 M7 | SESLRDAEKARRWCV |
| 8 | B4 M8 | SESLRDL [symbol] KARRWCV |
| 9 | B4 M9 | SESLRDLE [symbol] ARRWCV |
| 10 | B4 M10 | SESLRDLEKARRWCV |
| 11 | B4 M11 | SESLRDLEKA [symbol] RWCV |
| 12 | B4 M12 | SESLRDLEKAR [symbol] WCV |
| 13 | B4 M13 | SESLRDLEKARR [symbol] CV |
| 14 | B4 M14 | SESLRDLEKARRW [symbol] V |
| 15 | B4 M15 | SESLRDLEKARRWC [symbol] |

Number 1 above (B4 M1) is SEQ ID NO:56, number 2 (B4 M2) is SEQ ID NO:57, number 3 (B4 M3) is SEQ ID NO:58, number 4 (B4 M4) is SEQ ID NO:59, number 5 (B4 M5) is SEQ ID NO:60, number 6 (B4 M6) is SEQ ID NO:61, number 7 (B4 M7) is SEQ ID NO:62, number 8 (B4 M8) is SEQ ID NO:63, number 9 (B4 M9) is SEQ ID NO:64, number 10 (B4 M10) is SEQ ID NO:5, number 11 (B4 M11) is SEQ ID NO:66, number 12 (B4 M12) is SEQ ID NO:67, number 13 (B4 M13) is SEQ ID NO:68, number 14 (B4 M14) is SEQ ID NO:69, number 15 (B4 M15) is SEQ ID NO:70.

The alanine substitution mutants of C11 are shown below:

| NO: | Name | IDR mutant |
|---|---|---|
| 1 | C11 M1 | [symbol] FVAYTKDWCTKPKK |
| 2 | C11 M2 | S [symbol] VAYTKDWCTKPKK |
| 3 | C11 M3 | SF [symbol] AYTKDWCTKPKK |
| 4 | C11 M4 | SFVAYTKDWCTKPKK |
| 5 | C11 M5 | SFVA [symbol] TKDWCTKPKK |
| 6 | C11 M6 | SFVAY [symbol] KDWCTKPKK |
| 7 | C11 M7 | SFVAYT [symbol] DWCTKPKK |
| 8 | C11 M8 | SFVAYTK [symbol] WCTKPKK |
| 9 | C11 M9 | SFVAYTKD [symbol] CTKPKK |
| 10 | C11 M10 | SFVAYTKDW [symbol] TKPKK |
| 11 | C11 M11 | SFVAYTKDWC [symbol] KPKK |
| 12 | C11 M12 | SFVAYTKDWCT [symbol] PKK |
| 13 | C11 M13 | SFVAYTKDWCTK [symbol] KK |
| 14 | C11 M14 | SFVAYTKDWCTKP [symbol] K |
| 15 | C11 M15 | SFVAYTKDWCTKPK [symbol] |

Number 1 above (C11 M1) is SEQ ID NO:71, number 2 (C11 M2) is SEQ ID NO:72, number 3 (C11 M3) is SEQ ID NO:73, number 4 (C11 M4) is SEQ ID NO:7, number 5 (C11 M5) is SEQ ID NO:75, number 6 (C11 M6) is SEQ ID NO:76, number 7 (C11 M7) is SEQ ID NO:77, number 8 (C11 M8) is SEQ ID NO:78, number 9 (C11 M9) is SEQ ID NO:79, number 10 (C11 M10) is SEQ ID NO:80, number 11 (C11 M11) is SEQ ID NO:81, number 12 (C11 M12) is SEQ ID NO:82, number 13 (C11 M13) is SEQ ID NO:83, number 14 (C11 M14) is SEQ ID NO:84, number 15 (C11 M15) is SEQ ID NO:85.

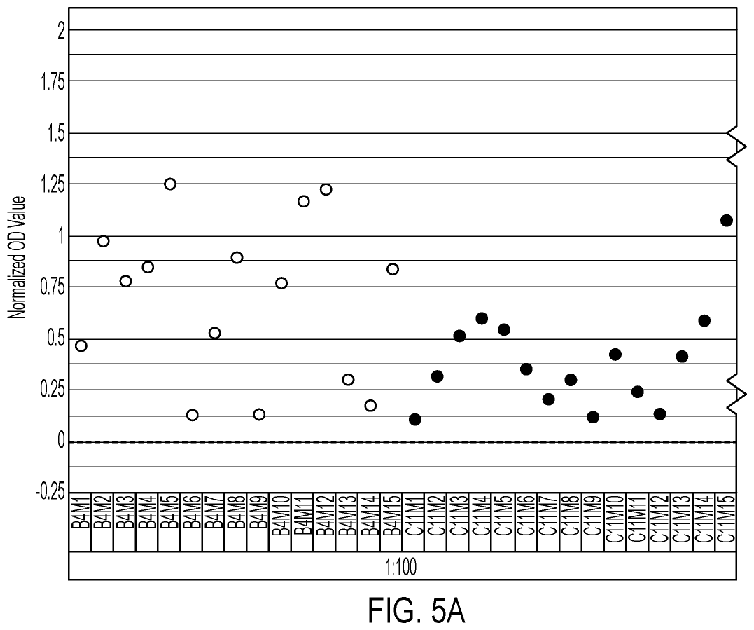

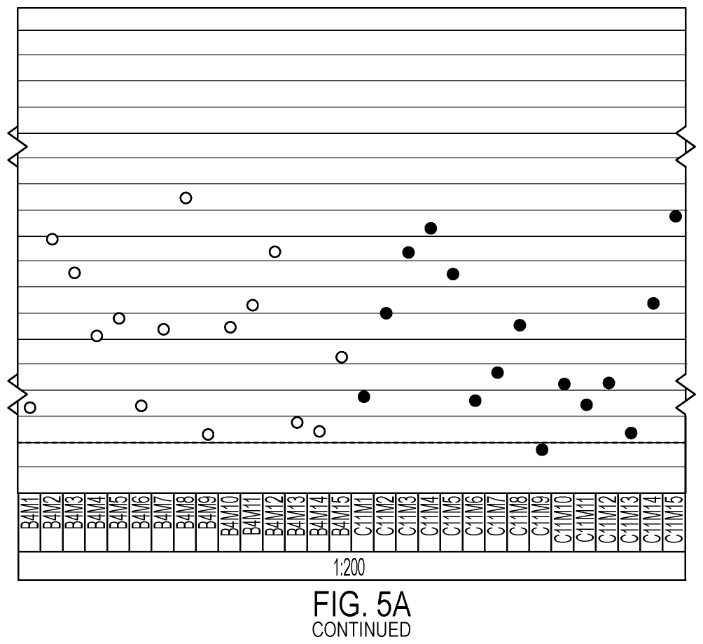

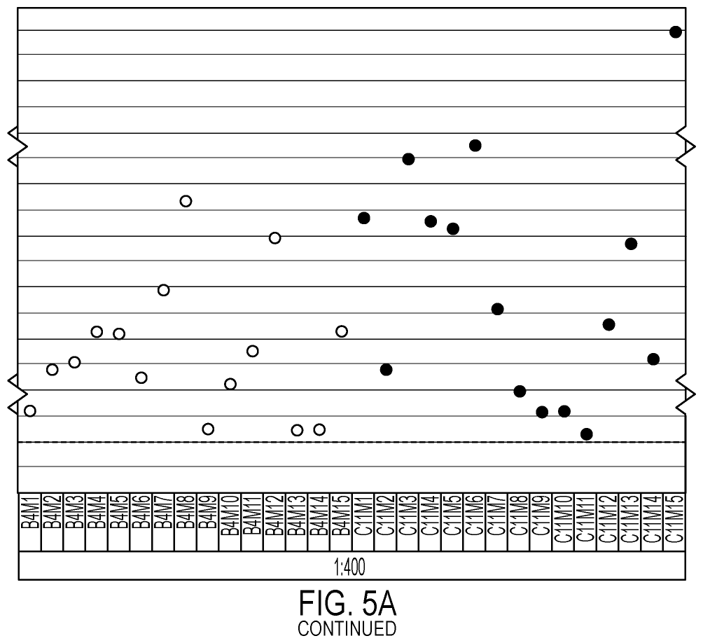

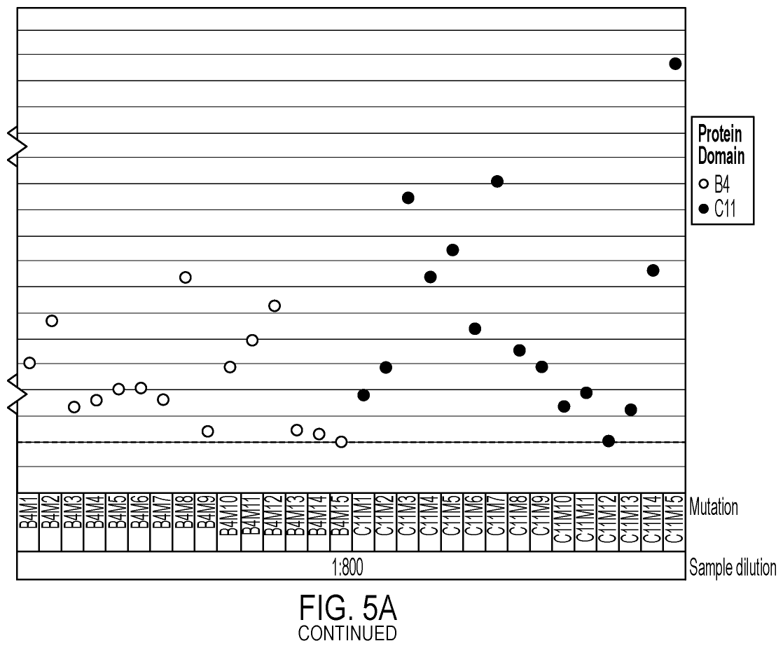

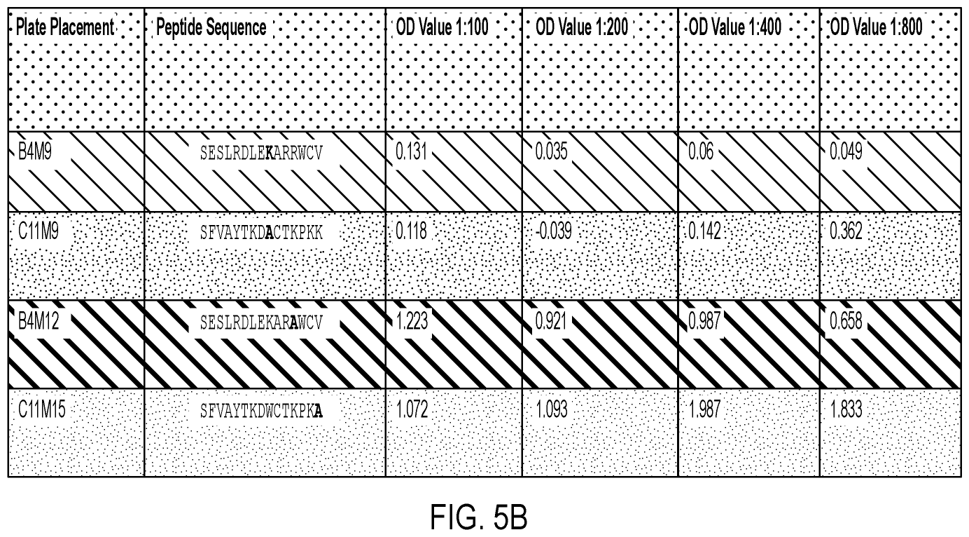

The peptides were tested with an indirect ELISA antibody assay for alanine scanning. Briefly, the peptides were coated onto neutravidin plates overnight (3X wash with plate washer) Samples (field negative and experimentally induced negative) are diluted 1:200, 1:400 and 1:800 are plated for 1 hr at room temperature (3 X Plate Washer). Secondary antibodies (goat anti-cat HRP) 1:5000 were coated for 1 hr RT and 5 X wash. Substrate was added and after 5 min stop solution added and read at 450. The results are shown in FIGS. 5A and 5B.

The B4M12 peptide, SESLRDLEKARAWCV (SEQ ID NO:67), showed a consistent high OD value indicating increased affinity for cat antibody. The B4M9 peptide, SESLRDLEAARRWCV (SEQ ID NO:64), showed a consistent low OD Value Indicating low affinity for cat antibody.

B4M9 and C11M9 both carry lysine to alanine substitutions that destroy binding activity. Therefore, these lysine residues should be retained to retain binding activity.

B4M12 and C11M15 both carry advantageous substitutions that increase observed OD Value and suggest enhanced antibody interaction. For B4M9 positively charged side chain change to a nonpolar hydrophobic side chain leads to a decrease in affinity for cat antibody suggesting that this change is important for the antibody peptide interaction. For B4M12 a negatively charged side chain change to nonpolar hydrophobic side chain leads to an increase in affinity for cat antibody.

TDX1362 and TDX1452 were tested in a multiplex ELISA assay. The feline serum samples tested are shown in Table 4.

TABLE 4

| Country of | Collection | # Samples | # Samples with |
|---|---|---|---|
| Origin | Year | Tested | Gender Data |
| Brazil | Pre-2020 | 80 | 80 |
| | 2020 | 322 | 168 |
| USA | Pre-2020 | 99 | |
| | 2020 | 920 | |

| | | |
|---|---|---|
| Totals | 1,421 | 248 |

TABLE 5

| Bead | | Bead | Conjugate | Biotinylated |
|---|---|---|---|---|
| Name | Marker | Type | Input | Conjugates |
| FIV | FIV peptide | Amino | 2.0 ug/mL | FIV peptide |
| FeLV | ADX-105 mAb | Carboxyl | 0.5 ug/mL | ADX-106 mAb |
| TDX- | TDX-1452 | Carboxyl | 1.0 ug/mL | TDX-1452 protein |
| 1452 | protein | | | |
| TDX- | TDX-1362 | Carboxyl | 1.0 ug/mL | TDX-1362 protein |
| 1362 | protein | | | |

TDX-1362 and TDX1452 were tested along with assays for FIV and FeLV in the multiplex assay. See Table 5. Briefly, recombinant TDX1452 and TDX1362 were conjugated to barcoded magnetic beads. Feline sera were added to the assay plates. Biotin labeled recombinant TDX1452 and TDX1362 were added to the assay plates. The labeled peptides were detected using streptavidin-PE. The results are shown in Table 6. The data indicates that sera can be tested for Mhf in multiplex with tests for FIV and FELV.

TABLE 6

| Calculation Method | # Samples | # Positive | Percent |
|---|---|---|---|
| (Cutoff = 250 MFI) | Tested | Samples | Positive |
| TDX-1362 Only | 1395 | 75 | 5.38% |
| TDX-1452 Only | 1394 | 61 | 4.38% |
| Both Markers | 1394 | 45 | 3.23% |

TABLE 7

| | # Samples | TDX-1362 | TDX-1452 | Dual |
|---|---|---|---|---|
| Gender | Tested | Positives | Positives | Positives |
| Male | 136 | 8.09% | 8.09% | 7.35% |
| Female | 112 | 4.46% | 0.89% | 0.89% |

The markers showed a 4-6% positive rate across the samples from the US and Brazil. Male cats showed a higher positive rate which is consistent with previous findings. Table 7. There was an increase in Mhf PCR positive rate in anemic, FIV or FeLV positive and male cats. Tables 8-10.

TABLE 8
Anemic Cats
Mhf PCR Positive Rates (2015-2020, n = 16,511)

| Anemia Status | Female | Male | Female and Male |
|---|---|---|---|
| No Anemia (HCT.28.1) | 0.7% | 2.6% | 1.9% |
| Non-Regenerative Anemia | 1.5% | 4.9% | 3.5% |
| (Reticulocyte count <50) | | | |
| Regenerative Anemia | 5.5% | 20.5% | 14.0% |
| Total | 1.8% | 6.0% | 4.4% |

TABLE 9
FIV Infected Cats
Mhf PCR Positive Rates (2015-2020, n = 33,119)

| FIV Status | Female | Male | Female and Male |
|---|---|---|---|
| Negative | 1.2% | 4.1% | 2.9% |
| Positive | 7.7% | 16.6% | 14.4% |
| Total | 1.3% | 4.7% | 3.3% |

TABLE 10
FeLV Infected Cats
Mhf PCR Positive Rates (2015-2020, n = 33,119)

| FeLV Status | Female | Male | Grand Total |
|---|---|---|---|
| Negative | 1.3% | 4.4% | 3.1% |
| Positive | 2.5% | 13.6% | 9.2% |
| Grand Total | 1.3% | 4.7% | 3.4% |

About 2/5 of FIV positive cats were Mhf positive. Total FeLV positive-46 FeLV/Mhf coinfection-17/46 (39%)

TABLE 11

| Country of | # Samples | # Positive | Percent |
|---|---|---|---|
| Origin | Tested | Samples | Positive |
| Brazil | 402 | 16 | 3.98% |
| USA | 1019 | 15 | 1.47% |

### EXAMPLE 4

M. haemofelis PCR positive and negative samples were screened by LCMS. TDX 1452 was identified as a possible candidate marker. Recombinant TDX1452 was expressed in E. coli and purified using an IMAC column.

An indirect ELISA assay was designed wherein the TDX1452 was immobilized to an Immulon IV plate. Briefly, the plates were coated with recombinant TDX1452 for an overnight period at 4° C. and the plates were washed with PBST and blocked with 2% BSA for 2-3 hours. The plates were washed again and stored at 4° C. for future use. The plates thawed at RT for 3-4 hours were used in the experiment. The serum samples were diluted with PRRS diluent and added to the plates and incubated for an hour at RT. The plates were washed again with PBST and the secondary antibody goat anti-IgG HRP diluted in enzyme conjugate diluent was added and incubated for 30 min at RT. The plates were washed with PBST and the substrate was added and read at 450 nm. Sera from experimentally infected M. haemofelis cats, sera from samples that were PCR positive for M. haemofelis, samples that were PCR negative for M. haemofelis, and samples from a feline general population were added to the assay plates. Labeled anti-feline IgG was added to the assay plates. The label was then detected.

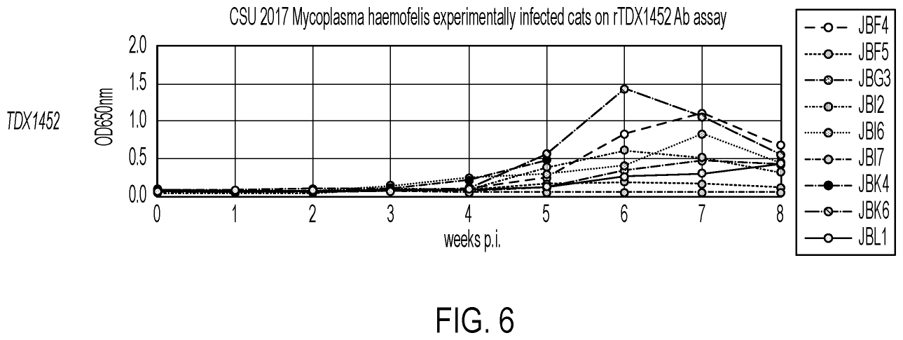

The indirect ELISA assay was used to test for antibody response in M. haemofelis experimentally infected cats over time. The recombinant protein was coated on an Immulon IV plate for an overnight period at 4° C., and the plate was blocked with blocking solution (1% BSA in 100 mM Tris pH 7.5). The plates were washed with 5×PBST, and the serum samples from experimentally infected cats were diluted with PRRS diluent and added to the plates, and kept for an hour at RT. The plates were washed again with 5×PBST, and the secondary antibody (Anti-Cat IgG H+L) was added to the plates and kept it for 30 minutes. The plates were washed, and the substrate was added and read at 450 nm. The indirect ELISA assay was able to detect anti-M. haemofelis in the sera samples as early as about 4.5 weeks post-infection. See FIG. 6.

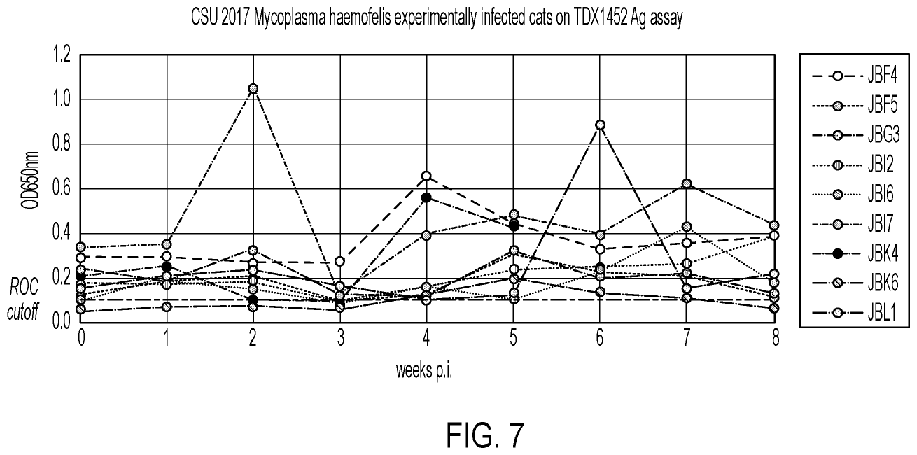

In another assay, streptavidin plates were coated with biotinylated anti-TDX1452 recombinant antigen polyclonal antibodies. Polyclonal antibodies against the recombinant TDX1452 protein were generated in rabbits using a standard protocol (SDIX, LLC, Newark, DE, USA). Briefly, preimmune sera were collected before the immunization of the rabbits. The animals were subcutaneously immunized with 200 μg of immunogen with Freund's complete adjuvant, and then a booster dose was provided on day 21, day 35, and day 50. Blood samples were collected on day 45, day 60, and day 70. Serum samples derived from the bloods were evaluated using an ELISA assay with recombinant antigen-coated plates to determine the antibody titer against the immunogen. Serially diluted anti-TDX1452 recombinant antigens were used as controls in plate assay. 50 mL undiluted serum/plasma sample was added to plate and incubated 30 min at room temp. 50 mL HRP-conjugated anti-TDX1452 pAbs were used for detection. The plates were incubated 30 min at room temp. 50 mL TMB was added to each well and the plates were incubated for 10 minutes at room temp. 50 mL stop solution was added and the plates were read at A650 nm. The results of testing on experimentally infected cats are shown in FIG. 7.

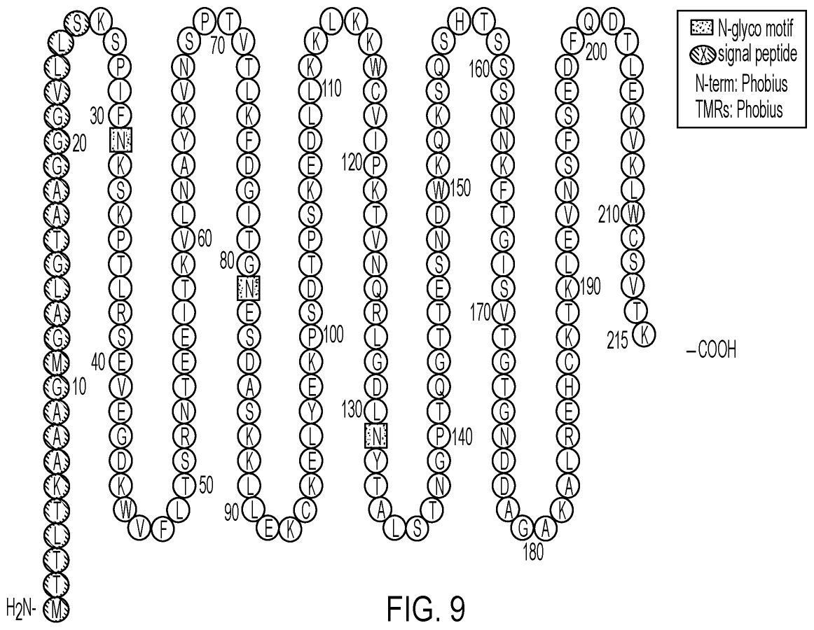

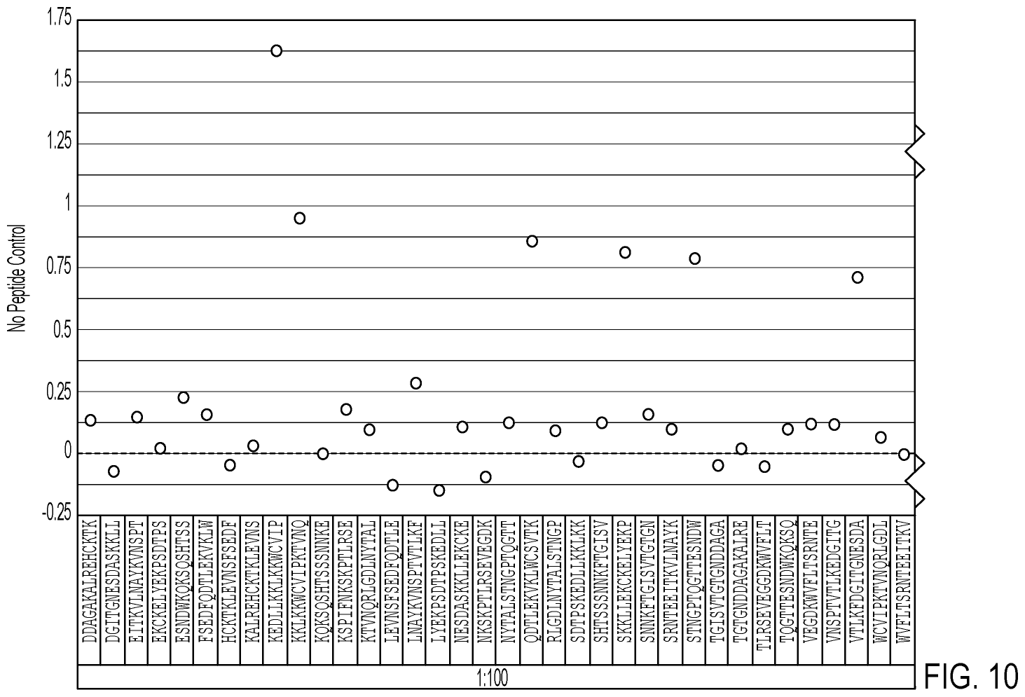

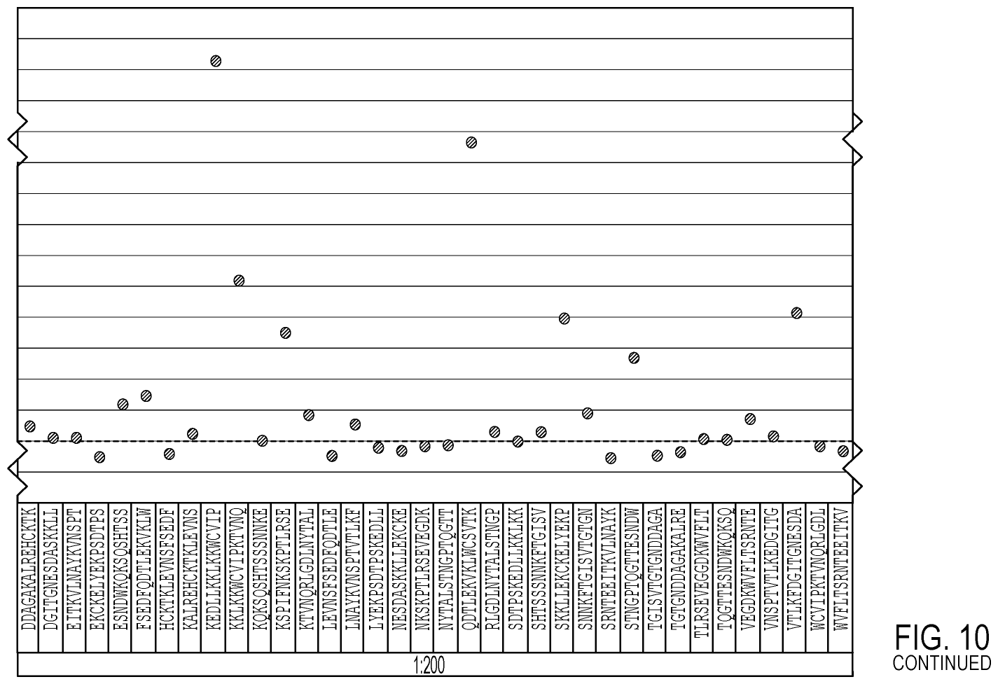

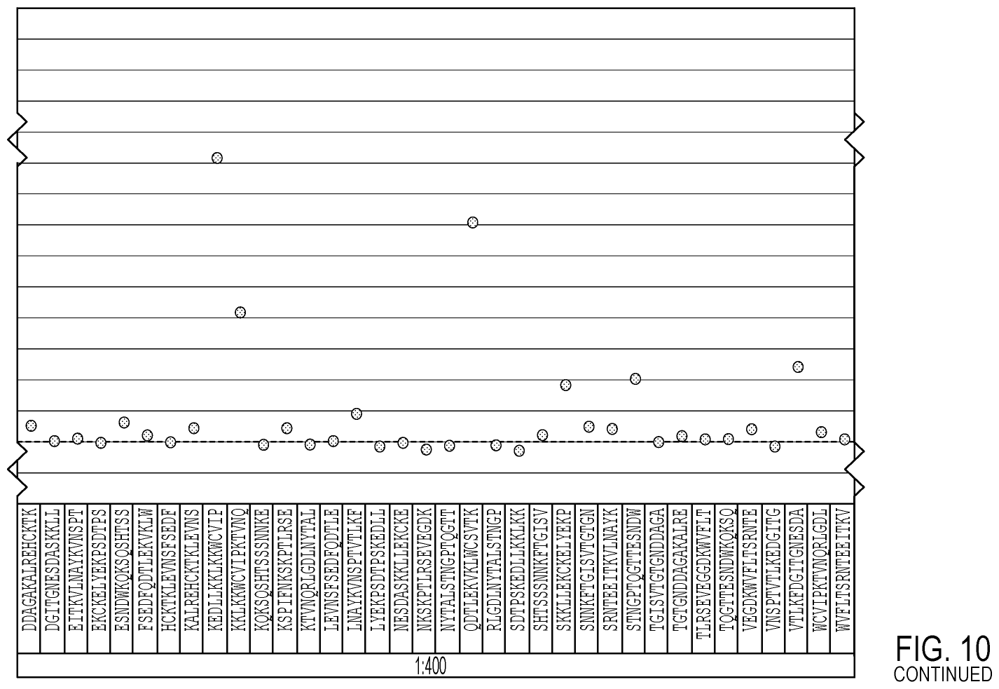

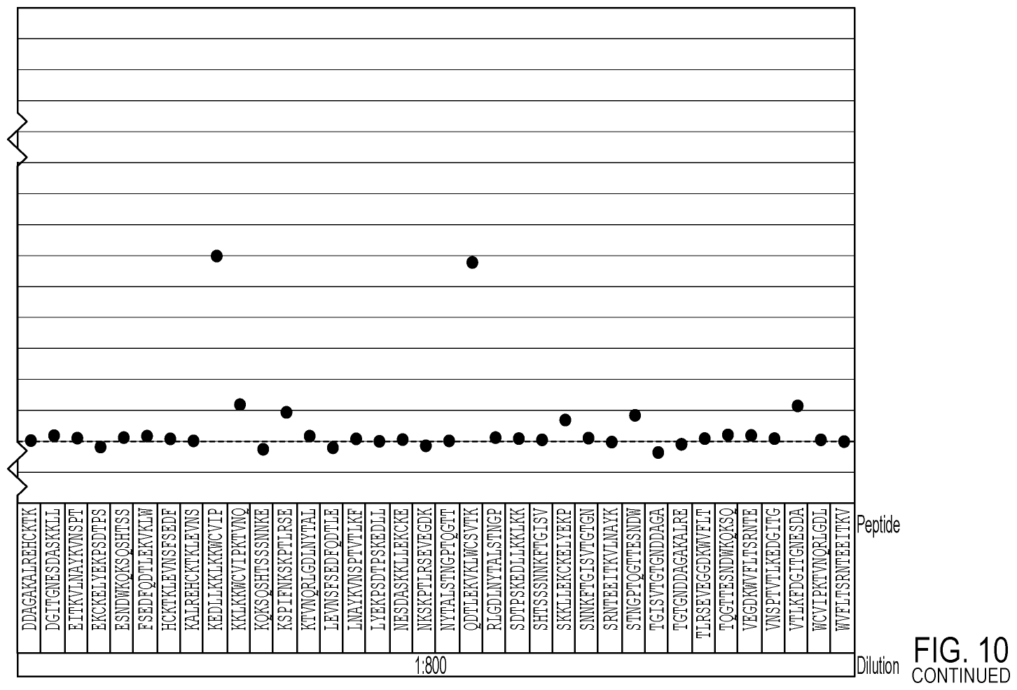

A series of 15 mer peptides overlapping by 10 amino acids of TDX1452 (FIG. 9) were generated to elucidate the immunodominant regions of the protein. FIG. 10.

Peptides B5, B6 and C12 demonstrated high affinity for cat antibodies.

B5
(SEQ ID NO: 1)
KEDLLKKLKKWCVIP
B6
(SEQ ID NO: 151)
KKLKKWCVIPKTVNQ
C12
(SEQ ID NO: 3)
QDTLEKVKLWCSVTK

The sequence overlap between B5 and B6 indicates that antibody from M. haemofelis infected cats binds within the polypeptide sequence that is shared by B5 and B6.

B5/B6:
(SEQ ID NO: 141)
KKLKKWCVIP

Peptides were coated onto neutravidin plates overnight (3X wash with plate washer). Serum samples (Feline Field Mhf Positive, Feline Field Mhf Negative, Rabbit, Feline Temporal Mhf infection; and SPF Negative) were diluted 1:200 and plated for 1 hr at room temperature (3 X Plate Washer). Secondary antibodies 1:5000 were coated for 1 hr RT. The plates were washed and substrate added. After 5 min the stop solution was added. The plates were read at 450 nm. B5, B6 and C12 showed a strong immunological response.

### EXAMPLE 5

A substitution of alanine for each amino acid position in 15-mer peptides were made. The immunodominant region, B5 and C12 were synthesized with these substitutions.

B5 alanine substitutions are shown below.

| Peptide | Peptide Code |
|---|---|
| KEDLLKKLKKWCVIP | B5 |
| (SEQ ID NO: 1) | |
| AEDLLKKLKKWCVIP | B5 M1 |
| (SEQ ID NO: 86) | |
| KADLLKKLKKWCVIP | B5 M2 |
| (SEQ ID NO: 87) | |
| KEALLKKLKKWCVIP | B5 M3 |
| (SEQ ID NO: 88) | |
| KEDALKKLKKWCVIP | B5 M4 |
| (SEQ ID NO: 89) | |
| KEDLAKKLKKWCVIP | B5 M5 |
| (SEQ ID NO: 90) | |
| KEDLLAKLKKWCVIP | B5 M6 |
| (SEQ ID NO: 91) | |
| KEDLLKALKKWCVIP | B5 M7 |
| (SEQ ID NO: 92) | |
| KEDLLKKAKKWCVIP | B5 M8 |
| (SEQ ID NO: 93) | |
| KEDLLKKLAKWCVIP | B5 M9 |
| (SEQ ID NO: 94) | |
| KEDLLKKLKAWCVIP | B5 M10 |
| (SEQ ID NO: 95) | |
| KEDLLKKLKKACVIP | B5 M11 |
| (SEQ ID NO: 96) | |
| KEDLLKKLKKWAVIP | B5 M12 |
| (SEQ ID NO: 97) | |
| KEDLLKKLKKWCAIP | B5 M13 |
| (SEQ ID NO: 98) | |
| KEDLLKKLKKWCVAP | B5 M14 |
| (SEQ ID NO: 99) | |
| KEDLLKKLKKWCVIA | B5 M15 |
| (SEQ ID NO: 100) | |

C12 alanine substitutions are show below.

| Peptide | Peptide Code |
|---|---|
| QDTLEKVKLWCSVTK | C12 |
| (SEQ ID NO: 3) | |
| ADTLEKVKLWCSVTK | C12 M1 |
| (SEQ ID NO: 101) | |
| QATLEKVKLWCSVTK | C12 M2 |
| (SEQ ID NO: 102) | |
| QDALEKVKLWCSVTK | C12 M3 |
| (SEQ ID NO: 103) | |
| QDTAEKVKLWCSVTK | C12 M4 |
| (SEQ ID NO: 104) | |
| QDTLAKVKLWCSVTK | C12 M5 |
| (SEQ ID NO: 105) | |
| QDTLEAVKLWCSVTK | C12 M6 |
| (SEQ ID NO: 106) | |
| QDTLEKAKLWCSVTK | C12 M7 |
| (SEQ ID NO: 107) | |
| QDTLEKVALWCSVTK | C12 M8 |
| (SEQ ID NO: 108) | |
| QDTLEKVKAWCSVTK | C12 M9 |
| (SEQ ID NO: 109) | |
| QDTLEKVKLACSVTK | C12 M10 |
| (SEQ ID NO: 110) | |
| QDTLEKVKLWASVTK | C12 M11 |
| (SEQ ID NO: 111) | |
| QDTLEKVKLWCAVTK | C12 M12 |
| (SEQ ID NO: 112) | |
| QDTLEKVKLWCSATK | C12 M13 |
| (SEQ ID NO: 113) | |
| QDTLEKVKLWCSVAK | C12 M14 |
| (SEQ ID NO: 114) | |
| QDTLEKVKLWCSVTA | C12 M15 |
| (SEQ ID NO: 115) | |

In positions where alanine was already present glycine was used. Critical amino acids can be detected if a large drop to OD is observed suggesting that the amino acid substituted was critical to antibody binding. This experiment also gives the opportunity for the identification of advantageous substitutions if an OD value is higher or increased compared to original peptide.

An indirect assay was used for epitope mapping of the alanine substituted B5 and C12 peptides. Peptides were biotinylated on the N terminus, coated onto neutravidin plates at 1 μg/mL, and incubated overnight at 4° C. Plates were washed 3 times with plate washer. The samples were incubated for 1 hr at room temperature (3 X Plate Washer). Secondary antibodies were coated on the plates for 1 hr RT and washed. Substrate was added and after 5 min a stop solution added. The plates were read at 450 nm.

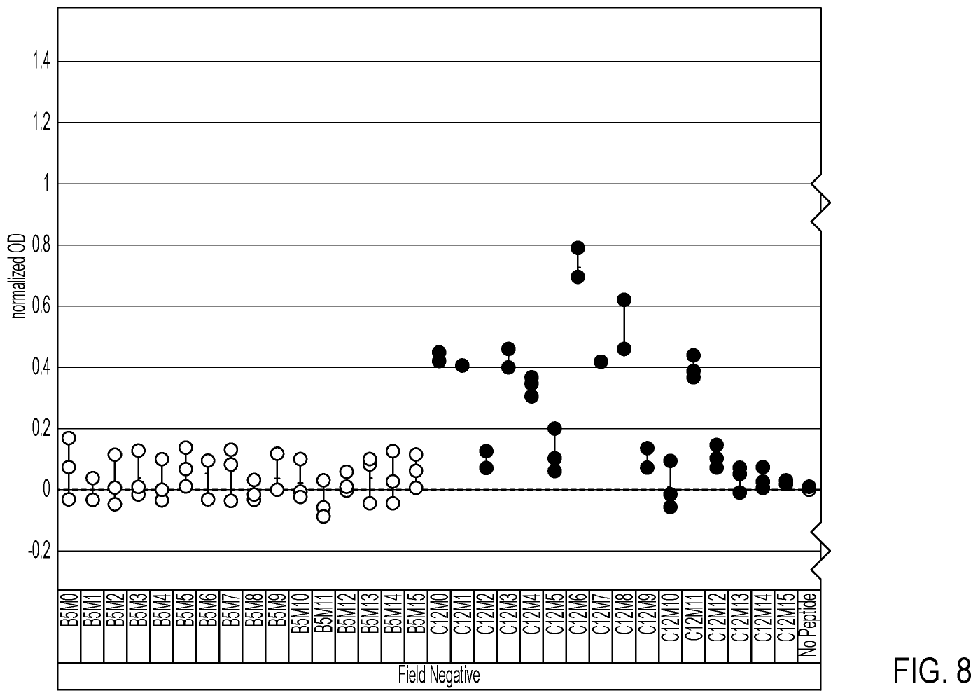

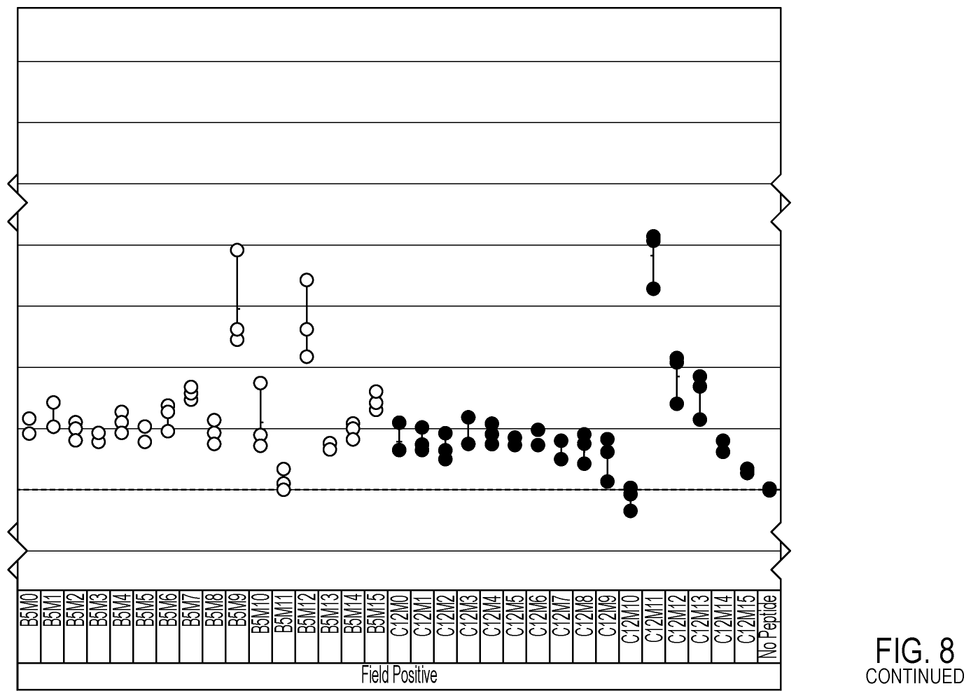

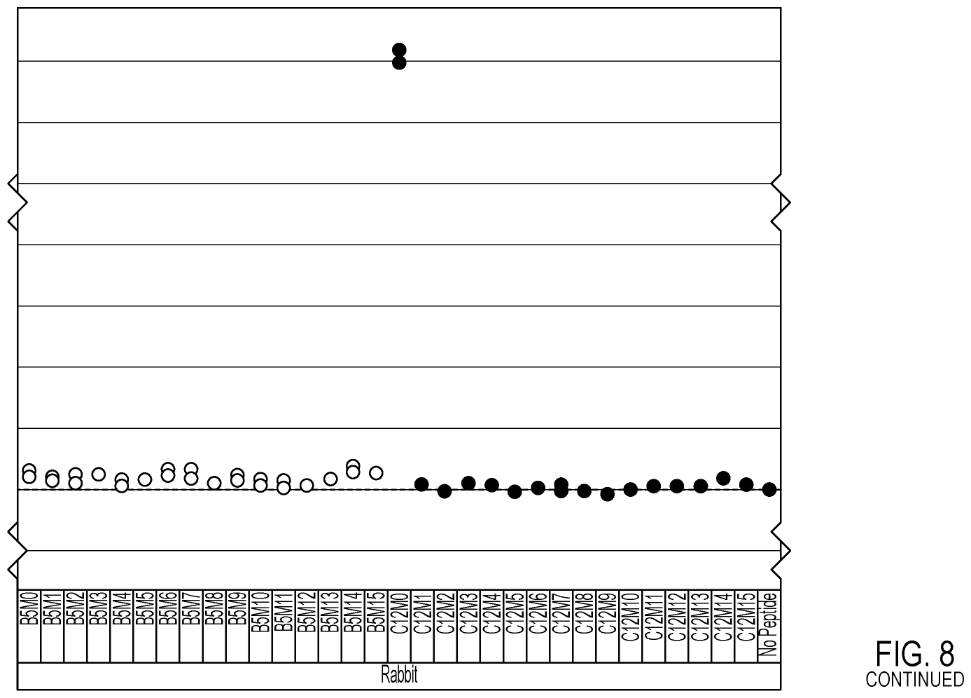

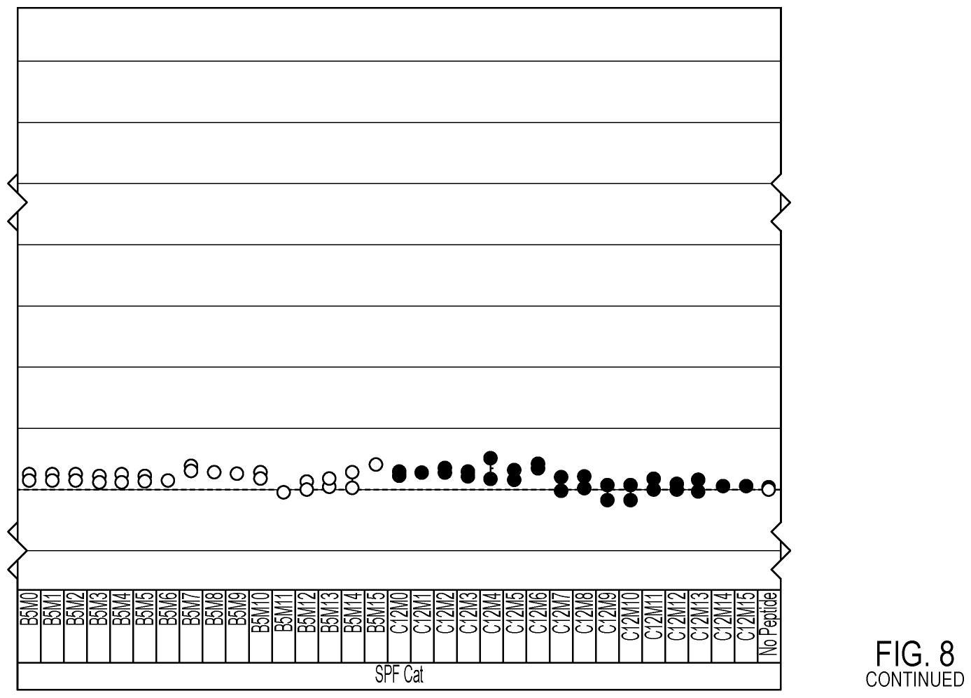

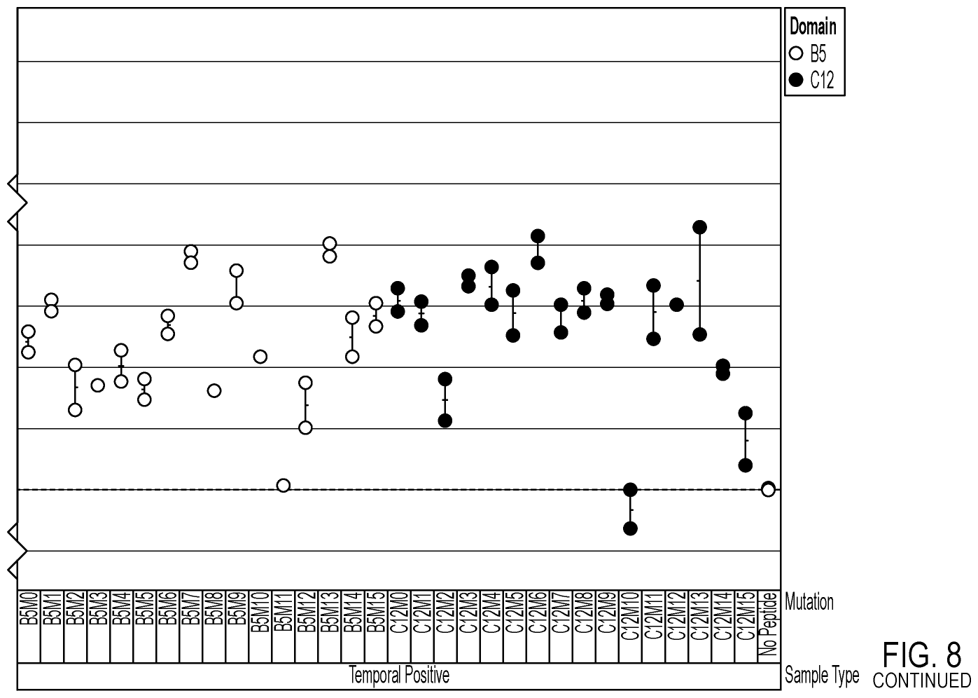

The results are shown in FIG. 8 and Table 12.

TABLE 12

| | | Normalized OD | Normalized Temporal |
|---|---|---|---|
| Mutation | Peptide | Field Positive | Positive |
| B5M0 | KEDLLKKLKKWCVIP | 0.19633 | 0.4785 |
| | SEQ ID NO: 1 | | |
| C12M0 | QDTLEKVKLWCSVTK | 0.155 | 0.616 |
| | SEQ ID NO: 3 | | |
| B5M11 | KEDLLKKLKKACVIP | 0.0233 | 0.011 |
| | SEQ ID NO: 96 | | |
| C12M10 | QDTLEKVKLACSVTK | −0.031 | −0.07 |
| | SEQ ID NO: 110 | | |
| B5M12 | KEDLLKKLKKWAVIP | 0.543 | 0.2715 |
| | SEQ ID NO: 97 | | |
| C12M11 | QDTLEKVKLWASVTK | 0.76033 | 0.576 |
| | SEQ ID NO: 111 | | |

The B5M12 peptide, KEDLLKKLKKWAVIP (SEQ ID NO:97), showed OD values higher than wildtype B5MO peptide, KEDLLKKLKKWCVIP (SEQ ID NO:1), indicating that the B5M12 (C>A) mutation increased binding with cat anti-Mhf antibody. The B5M11 peptide, KEDLLKKLKKACVIP (SEQ ID NO:96), showed lower OD values than the wildtype B5MO peptide, indicating that the substituted amino acid residue (tryptophan) is important for the antibody-peptide interaction and should be retained to retain binding activity.

The C12M11 peptide, QDTLEKVKLWASVTK (SEQ ID NO:111), showed OD values higher than wildtype C12MO peptide, QDTLEKVKLWCSVTK (SEQ ID NO:3), indicating that the C12M11 (C>A) mutation increased binding with cat anti-Mhf antibody. The C12M10 peptide, QDTLEKVKLACSVTK (SEQ ID NO: 110), showed lower OD values than the wildtype C12MO peptide, indicating that the substituted amino acid residue (tryptophan) is important for the antibody-peptide interaction and should be retained to retain binding activity.

1. One or more polypeptides comprising one or more of:
(i) a polypeptide as set forth in SEQ ID NO:1, 2, 3, 4, 5, 6, 7, 8, 37, 54, 74, 97, 111, 140, 141, or 151;
(ii) a polypeptide comprising SEQ ID NO:1, wherein:
(a) Wat position 11 is retained and 1, 2, or 3 other amino acids are substituted with a conservative amino acid substitution;
(b) W at position 11 is retained and conservative amino acid substitutions are at 1, 2, or 3 of the following positions: 1, 2, 3, 4, 5, 6, 7, 8, 9, 10, 12, 13, 14, or 15;
(c) W at position 11 is retained and conservative amino acid substitutions are at 1, 2, or 3 of the following positions: 2, 3, 4, 6, 7, 8, or 14; or
(d) W at position 11 is retained and position 12 is substituted with A, and optionally conservative amino acid substitutions are at 1, 2, or 3 of the following positions: 1, 2, 3, 4, 5, 6, 7, 8, 9, 10, 13, 14, or 15;
(iii) a polypeptide comprising SEQ ID NO:2, wherein:
(a) W at position 11 is retained and 1, 2, 3 other amino acids are substituted with a conservative amino acid substitution;
(b) W at position 11 is retained and conservative amino acid substitutions are at 1, 2, or 3 of the following positions: 1, 2, 3, 4, 5, 6, 7, 8, 9, 10, 12, 13, 14, or 15; or
(c) W at position 11 is retained and conservative amino acid substitutions are at 1, 2, or 3 of the following positions: 2, 3, 4, 6, 7, 8, or 14;
(iv) a polypeptide comprising SEQ ID NO:3 wherein:
(a) W at position 10 is retained and 1, 2, 3 other amino acids are substituted with a conservative amino acid substitution;
(b) W at position 10 is retained and conservative amino acid substitutions are at 1, 2, or 3 of the following positions: 1, 2, 3, 4, 5, 6, 7, 8, 9, 11, 12, 13, 14, or 15; or
(c) W at position 11 is retained and conservative amino acid substitutions are at 1, 2, or 3 of the following positions: 1, 3, 5, 6, 7, 8, 14 or 15;
(v) a polypeptide comprising SEQ ID NO:4, wherein:
(a) W at position 10 is retained and 1, 2, 3 other amino acids are substituted with a conservative amino acid substitution;
(b) W at position 10 is retained and conservative amino acid substitutions are at 1, 2, or 3 of the following positions: 1, 2, 3, 4, 5, 6, 7, 8, 9, 11, 12, 13, 14, or 15; or
(c) W at position 11 is retained and conservative amino acid substitutions are at 1, 2, or 3 of the following positions: 1, 3, 5, 6, 7, 8, 14 or 15;
(vi) a polypeptide comprising SEQ ID NO:5, wherein:
(a) K at position 9 and Wat position 13 are retained and 1, 2, 3 other amino acids are substituted with a conservative amino acid substitution;
(b) K at position 9 and W at position 13 are retained and conservative amino acid substitutions are at 1, 2, or 3 of the following positions: 1, 2, 3, 4, 5, 6, 7, 8, 10, 11, 12, 14, or 15; or
(c) K at position 9 and W at position 13 are retained and conservative amino acid substitutions are at 1, 2, or 3 of the following positions: 2, 3, 4, or 5;
(vii) a polypeptide comprising SEQ ID NO:6, wherein:
(a) K at position 9 and the W at position 13 are retained and 1, 2, 3 other amino acids are substituted with a conservative amino acid substitution;
(b) K at position 9 and W at position 13 are retained and conservative amino acid substitutions are at 1, 2, or 3 of the following positions: 1, 2, 3, 4, 5, 6, 7, 8, 10, 11, 12, 14, or 15; or
(c) K at position 9 and W at position 13 are retained and conservative amino acid substitutions are at 1, 2, or 3 of the following positions: 2, 3, 4, or 5;
(viii) a polypeptide comprising SEQ ID NO:7, wherein:
(a) K at position 7, D at position 8, and W at position 9 are retained and 1, 2, 3 other amino acids are substituted with a conservative amino acid substitution;
(b) K at position 7, D at position 8, and W at position 9 are retained and conservative amino acid substitutions are at 1, 2, or 3 of the following positions: 1, 2, 3, 4, 5, 6, 10, 11, 12, 13, 14, or 15; or
(c) K at position 7, D at position 8, and W at position 9 are retained and conservative amino acid substitutions are at 1, 2, or 3 of the following positions: 3, 4, or 15.
(ix) a polypeptide comprising SEQ ID NO:8, wherein
(a) D at position 8 and W at position 9 are retained and 1, 2, 3 other amino acids are substituted with a conservative amino acid substitution;
(b) D at position 8 and W at position 9 are retained and conservative amino acid substitutions are at 1, 2, or 3 of the following positions: 1, 2, 3, 4, 5, 6, 7, 10, 11, 12, 13, 14, or 15; or
(c) D at position 8 and the W at position 9 are retained and conservative amino acid substitutions are at 1, 2, or 3 of the following positions: 3, 4, 7, or 15;
(x) a polypeptide comprising SEQ ID NO:10;
(xi) a polypeptide having 95% or more sequence identity to SEQ ID NO: 10, wherein amino acids 75-89, 170-184, or 75-89, and 170-184 are retained;
(xii) a polypeptide comprising SEQ ID NO: 12;
(xiii) a polypeptide having 95% or more sequence identity to SEQ ID NO:12, wherein amino acids 82-96, 177-191, or 82-96 and 177-191 are retained;
(xiv) a polypeptide comprising SEQ ID NO:9 or SEQ ID NO: 11;
(xv) a polypeptide comprising a polypeptide comprising SEQ ID NO:141, wherein:
(a) W at position 6 is retained and 1, 2, or 3 other amino acids are substituted with a conservative amino acid substitution;
(b) W at position 6 is retained and conservative amino acid substitutions are at 1, 2, or 3 of the following positions: 1, 2, 3, 4, 5, 7, 8, 9, or 10; or
(c) Wat position 6 is retained and position 7 is substituted with A, and optionally conservative amino acid substitutions are at 1, 2, or 3 of the following positions: 1, 2, 3, 4, 5, 8, 9, or 10;
(xvi) a polypeptide comprising a polypeptide comprising SEQ ID NO: 151, wherein:
(a) Wat position 6 is retained and 1, 2, or 3 other amino acids are substituted with a conservative amino acid substitution;
(b) W at position 6 is retained and conservative amino acid substitutions are at 1, 2, or 3 of the following positions: 1, 2, 3, 4, 5, 7, 8, 9, 10, 11, 12, 13, or 14; or
(c) Wat position 6 is retained and position 7 is substituted with A, and optionally conservative amino acid substitutions are at 1, 2, or 3 of the following positions: 1, 2, 3, 4, 5, 8, 9, 10, 11, 12, 13, or 14;
(xvii) a polypeptide comprising a polypeptide comprising SEQ ID NO:97, wherein:
(a) W at position 11 is retained and 1, 2, or 3 other amino acids are substituted with a conservative amino acid substitution;
(b) W at position 11 is retained and conservative amino acid substitutions are at 1, 2, or 3 of the following positions: 1, 2, 3, 4, 5, 6, 7, 8, 9, 10, 12, 13, 14, or 15; or
(c) Wat position 11 is retained and position 12 is substituted with A, and optionally conservative amino acid substitutions are at 1, 2, or 3 of the following positions: 1, 2, 3, 4, 5, 8, 9, 10, 13, 14, or 15;
(xviii) a polypeptide comprising SEQ ID NO:74, wherein:
(a) K at position 4 and W at position 8 are retained and 1, 2, 3 other amino acids are substituted with a conservative amino acid substitution; or
(b) K at position 4 and W at position 8 are retained and conservative amino acid substitutions are at 1, 2, or 3 of the following positions: 1, 2, 3, 5, 6, 7, or 10;
(xix) a polypeptide comprising SEQ ID NO:37, wherein:
(a) K at position 4 and W at position 8 are retained and 1, 2, 3 other amino acids are substituted with a conservative amino acid substitution; or
(b) K at position 4 and W at position 8 are retained and conservative amino acid substitutions are at 1, 2, or 3 of the following positions: 1, 2, 3, 5, 6, 7, 11, 12, 13, 14, or 15;
(xx) a polypeptide comprising SEQ ID NO:54, wherein:
(a) K at position 12, D at position 13, and W at position 14 are retained and 1, 2, 3 other amino acids are substituted with a conservative amino acid substitution;
(b) K at position 12, D at position 13, and W at position 14 are retained and conservative amino acid substitutions are at 1, 2, or 3 of the following positions: 1, 2, 3, 4, 5, 6, 7, 8, 9, 10,
(c) Kat position 12, D at position 13, and Wat position 14 are retained and conservative amino acid substitutions are at 1, 2, or 3 of the following positions: 7 or 8;
(xxi) a polypeptide comprising SEQ ID NO:140, wherein:
(a) K at position 7, D at position 8, and W at position 9 are retained and 1, 2, 3 other amino acids are substituted with a conservative amino acid substitution;
(b) K at position 7, D at position 8, and W at position 9 are retained and conservative amino acid substitutions are at 1, 2, or 3 of the following positions: 1, 2, 3, 4, 5, 6, or 10; or
(c) K at position 7, D at position 8, and W at position 9 are retained and conservative amino acid substitutions are at 1, 2, or 3 of the following positions: 3 or 4.
(xxii) a polypeptide comprising SEQ ID NO:111 wherein:
(a) W at position 10 is retained and 1, 2, 3 other amino acids are substituted with a conservative amino acid substitution;
(b) W at position 10 is retained and conservative amino acid substitutions are at 1, 2, or 3 of the following positions: 1, 2, 3, 4, 5, 6, 7, 8, 9, 11, 12, 13, 14, or 15; or
(c) W at position 11 is retained and conservative amino acid substitutions are at 1, 2, or 3 of the following positions: 1, 3, 5, 6, 7, 8, 14 or 15.

2. A fusion protein comprising two, three, four, five, six, seven or more polypeptides having 90% or more sequence identity to the one or more polypeptides of claim 1.

3. A polypeptide having less than 190 total amino acids and comprising 90% or more sequence identity to a polypeptide as set forth in SEQ ID NO: 10 or 12.

4. (canceled)

5. (canceled)

6. (canceled)

7. (canceled)

8. The one or more polypeptides of claim 1, wherein the one or more polypeptides are present in an immunocomplex with one or more antibodies that specifically bind to a polypeptide as set forth in SEQ ID NO: 1, 2, 3, 4, 5, 6, 7, 8, 9, 10, 11, 12, 37, 54, 74, 97, 111, 140, 141, or 151.

9. The one or more polypeptides of claim 1, wherein the one or more polypeptides comprise one or more secretory signal sequences, one or more epitope tags, or one or more secretory signal sequences and one or more epitope tags.

10. A method of detecting anti-Mycoplasma haemofelis or anti-Mycoplasma haemocanis antibodies or specific binding fragments thereof comprising (a) contacting a test sample with the one or more polypeptides of claim 1; and (b) detecting complexes of anti-Mycoplasma haemofelis or anti-Mycoplasma haemocanis antibodies or specific binding fragments thereof and the one or more polypeptides.

11. (canceled)

12. The method of claim 10, wherein the complexes are detected using one or more secondary antibodies or specific binding fragments thereof that specifically bind anti-Mycoplasma haemofelis or anti-Mycoplasma haemocanis antibodies or specific binding fragments thereof.

13. (canceled)

14. The method of claim 10, wherein the complexes are detected using one or more detector polypeptides wherein the one or more detector polypeptides are the one or more polypeptides of claim 1.

15. (canceled)

16. A method for diagnosing a disease caused by Mycoplasma haemofelis or Mycoplasma haemocanis antibodies in a subject by (a) contacting a test sample with the one or more polypeptides of claim 1; and (b) detecting complexes of anti-Mycoplasma haemofelis or anti-Mycoplasma haemocanis antibodies or specific binding fragments thereof and the one or more polypeptides.

17. The method of claim 16, wherein the subject has been infected with Mycoplasma haemofelis or Mycoplasma haemocanis for less than 15 days.

18. The method of claim 16, further comprising comparing an amount of the complexes in the sample to a control sample or control standard, wherein elevated levels of the complexes as compared to the control sample or control standard is an indication of a disease caused by Mycoplasma haemofelis or Mycoplasma haemocanis.

19. The method of claim 16, further comprising administering a treatment for a disease caused by Mycoplasma haemofelis or Mycoplasma haemocanis where the complexes are detected.

20. (canceled)

21. (canceled)

22. (canceled)

23. The method of claim 10, wherein the anti-Mycoplasma haemofelis or anti-Mycoplasma haemocanis antibodies or specific binding fragments thereof are detected by a competitive immunoassay, a sandwich immunoassay, an enzyme-linked immunosorbent assay (ELISA), an immunohistochemical assay, a turbidimetric immunoassay, a particle-enhanced turbidimetric immunoassay, a radioimmunoassay (RIA), a fluorescent immunosorbent assay (FIA), a multiplex immunoassay, a protein/peptide array immunoassay, a solid phase radioimmunoassay (SPRIA), an indirect immunofluorescence assay (IIF), a chemiluminescent immunoassay (CIA), a particle based multianalyte test (PMAT), a dot blot assay, a western blot assay, surface plasmon resonance (SPR), isothermal titration calorimetry (ITC), microscale thermophoresis (MST), biolayer interferometry, or grating-coupled interferometry.

24. The method of claim 23, wherein the multiplex assay additionally tests for feline leukemia virus (FeLV), feline-immunodeficiency virus (FIV), or both FeLV and FIV.

25. A kit for diagnosing a disease caused by Mycoplasma haemofelis or Mycoplasma haemocanis, the kit comprising:
(a) the one or more polypeptides of claim 1, wherein the polypeptide is not naturally occurring; and
(b) one or more reagents that facilitate binding of the one or more polypeptides to anti Mycoplasma haemofelis or anti-Mycoplasma haemocanis antibodies or specific binding fragments thereof present in a test sample.

26. A method for treating a disease caused by Mycoplasma haemofelis or Mycoplasma haemocanis antibodies in a subject by (a) contacting a test sample with the one or more polypeptides of claim 1; (b) detecting complexes of anti-Mycoplasma haemofelis or anti-Mycoplasma haemocanis or specific binding fragments thereof and the one or more polypeptides; (c) treating the subject with one or more antibiotics where complexes of anti-Mycoplasma haemofelis or anti-Mycoplasma haemocanis antibodies or specific binding fragments thereof and the one or more polypeptides are detected.

27. The method of claim 26, wherein the subject has been infected with Mycoplasma haemofelis or Mycoplasma haemocanis for less than 15 days.

28. The method of claim 26, further comprising comparing an amount of the complexes in the sample to a control sample or control standard, wherein elevated levels of the complexes as compared to the control sample or control standard is an indication of a disease caused by Mycoplasma haemofelis or Mycoplasma haemocanis.

29. (canceled)

30. (canceled)

31. (canceled)

32. The method of claim 26, wherein the anti-Mycoplasma haemofelis or anti-Mycoplasma haemocanis antibodies or specific binding fragments thereof are detected by a competitive immunoassay, a sandwich immunoassay, an enzyme-linked immunosorbent assay (ELISA), an immunohistochemical assay, a turbidimetric immunoassay, a particle-enhanced turbidimetric immunoassay, a radioimmunoassay (RIA), a fluorescent immunosorbent assay (FIA), a multiplex immunoassay, a protein/peptide array immunoassay, a solid phase radioimmunoassay (SPRIA), an indirect immunofluorescence assay (IIF), a chemiluminescent immunoassay (CIA), a particle based multianalyte test (PMAT), a dot blot assay, a western blot assay, surface plasmon resonance (SPR), isothermal titration calorimetry (ITC), microscale thermophoresis (MST), biolayer interferometry, or grating-coupled interferometry.

33. A polynucleotide encoding the one or more polypeptides of claim 1.
